(19) United States
(12) Patent Application Publication
(10) Pub. No.: US 2010/0017143 A1
(43) Pub. Date: Jan. 21, 2010

(54) GESTATIONAL AGE DEPENDENT PROTEOMIC CHANGES OF HUMAN MATERNAL SERUM FOR MONITORING MATERNAL AND FETAL HEALTH

(75) Inventors: SRINIVASA R. NAGALLA, HILLSBORO, OR (US); JUHA RASANEN, OULU (FI); MICHAEL GRAVETT, SEATTLE, WA (US)

Correspondence Address: Goodwin Procter LLP;Attn: Patent Administrator, 135 Commonwealth Drive, Menlo Park, CA, 94025-1105, US

(73) Assignee: PROTEOGENIX, INC., COSTA MESA, CA (US)

(21) Appl. No.: 12/363,651

(22) Filed: Jan. 30, 2009

Related U.S. Application Data
(60) Provisional application No. 61/024,865, filed on Jan. 30, 2008.

(51) Int. Cl. G06F 19/00; G01N 33/48; G06G 7/48

(52) U.S. Cl. 702/19; 703/11

(57) ABSTRACT

The present invention concerns a global maternal serum proteome map and its changes during healthy gestation. Accordingly, the present invention provides an important tool for plasma-based maternal-fetal diagnostics.

### RELATED APPLICATION

This application claims priority under 35 U.S.C. §119(e) to U.S. provisional application No. 61/024,865, filed Jan. 30, 2008, the entire contents of which are incorporated herein by reference.

### BACKGROUND OF THE INVENTION

1. Field of the Invention

The present invention concerns a global maternal serum proteome map and its changes during healthy gestation. Accordingly, the present invention provides an important tool for plasma-based maternal-fetal diagnostics.

2. Description of the Related Art

Maternal plasma plays an important role during implantation, gestation and parturition. Insulin-like growth factors, their binding substrates (such as IGF-I and IGFBP-1) and cytokines present in maternal serum aid in embryonic implantation {Slater, 1999 #94; Sharkey, 1998 #95}. Angiogenic factors such as vascular endothelial growth factor and placental growth factor are involved in vascular remodeling of spiral arteries during pregnancy, which is critical for proper placental implantation {Muller, 2006 #89}. Maternal serum supplies all the necessary vitamins {Salle, 2000 #74; Bohles, 1997 #75}, minerals {Favier, 1990 #70; Pitkin, 1985 #73; Spatling, 1989 #72}, carbohydrates, lipids {Coleman, 1986 #76}, and amino acids {Battaglia, 1992 #78; Regnault, 2002 #77} to the developing fetus. During gestation, maternal serum also contains several placental proteins like human chorionic gonadotropin subunit β (βHCG), chorionic somatomammotrophin hormone (CSH), various forms of pregnancy-associated-β-1-glycoproteins (PSG), and pregnancy-associated proteins (PAPP-A, PAPP-B and PAPP-C etc.) that aid in fetal development {Grudzinskas, 1982 #79}. Some of these placental proteins (such as βHCG) are known to prevent maternal immuno-rejection of fetus {Knobloch, 1988 #96}. The blood coagulation and fibrinolysis systems of maternal plasma also change rapidly during gestation {Holmes, 2005 #103} and parturition {Schander, 1979 #97}. Maternal and/or placental pathologies (such as preeclampsia, Down syndrome, and preterm birth etc.) effect the composition and dynamics of maternal plasma {Cross, 2003 #88}. Abnormal levels of placental proteins such as PAPP-A and βHCG have been indicative of fetal disorders like aneuploidy {Malone, 2005 #84; Breathnach, 2007 #85} and obstetric complications like preterm birth {Dugoff, 2005 #87; Dolan, 2007 #86}. There are also reports of fetal mRNA {Steele, 1996 #81; Bianchi, 2004 #82} and proteins {Van Lith, 1991 #80} circulating in maternal plasma during fetal distress or presence of placental pathology {Wataganara, 2004 #83; Bianchi, 2004 #82}. Abnormal levels of maternal serum proteins like endothelin {Slowinski, 2002 #90}, soluble forms-like tyrosine kinase-1 (sFlt-1) {Lockwood, 2007 #92}, angiotensin-II {Xia, 2007 #93} etc. are associated with preeclampsia. In lieu of the above facts, maternal serum undergoes complex physiological changes during both normal and abnormal gestations. A systematic investigation of maternal serum protein changes during healthy gestation is essential for the development of next generation maternal-fetal protein biomarker based diagnostics.

Serum is a highly studied body fluid in field of proteomics. There have been several concerted {States, 2006 #68; Anderson, 2004 #67} and individual {Pieper, 2003 #98; Pieper, 2003 #99} efforts to extensively sequence the human plasma proteome. The human plasma proteome map is derived from a diverse population containing a majority of non-maternal samples. There has been only a single study {Michel, 2006 #101} to date that has exclusively sequenced a total of 79 plasma proteins from a single maternal subject. The total number of proteins identified in that study is well short of dynamic range of serum that could be probed with current proteomics technology. Thus, a unique maternal serum proteome map and its overlap with known serum proteome are still incomplete.

Amniotic fluid (AF) is an extensively sequenced maternal body fluid {Cho, 2007 #64; Tsangaris, 2006 #100; Michel, 2006 #101; Park, 2006 #102}. A total of 47 AF proteins are also found in maternal serum {Michel, 2006 #101}. AF is also known to change during gestation, just like maternal serum. There had been efforts to probe the dynamics of AF during normal gestation {Michaels, 2007 #63}. However, there have been no studies that have given similar treatment for maternal serum during gestation.

In this study, maternal serum was collected from a total of 44 healthy human subjects during their first, second and third trimesters, respectively. Pooled serum from each of the three trimesters was subjected to two-dimensional liquid chromatography (2-DLC) based tandem mass spectrometry. Sequenced maternal plasma from this study was functionally annotated and quantitatively compared to known plasma and AF proteomes. Maternal plasma proteins that are differentially expressed between all trimesters were identified using label-free quantitation. Proteins that showed a continuous expression change from first to third trimester are identified using label-free trend analysis. Selected biomarkers from label-free quantitation were validated with enzyme-linked immunosorbent assay (ELISA). The result of this study is derivation of a global maternal serum proteome map and its changes (protein expression) during healthy gestation, which forms a basis for next generation plasma based maternal-fetal diagnostics.

### SUMMARY OF THE INVENTION

In one aspect, the present invention concerns a proteomic profile of healthy maternal serum.

In another aspect, the present invention concerns a proteomic profile of healthy maternal serum in the first trimester of pregnancy.

In one embodiment, such proteomic profile comprises at least one characteristic expression signature present exclusively during the first trimester of pregnancy.

In another embodiment, the characteristic expression signature indicates upregulation of at least one protein selected from the group consisting of chorionic somatomammotropin hormone (P01243), pappalysin-1 (Q13219), pregnancy-specific β-1-glycoprotein 2 (P11465), apolipoprotein C-III (P02656), apolipoprotein A (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), apolipoprotein(a) (P08519), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-1(XVII) chain (Q9UMD9), leucyl-cystinyl aminopeptidase (Q9UIQ6), collagen α-1(I) chain (Q15201), and macrophage colony-stimulating factor (P07333).

In a further embodiment, the characteristic expression signature indicates upregulation of at least two of such proteins.

In a still further embodiment, the characteristic expression signature indicates upregulation of at least three of such proteins.

In another embodiment, the characteristic expression signature indicates upregulation of all of the listed proteins.

In a different aspect, the invention concerns a proteomic profile of healthy maternal serum in the second trimester of pregnancy.

In one embodiment, the proteomic profile comprises at least one characteristic expression signature present exclusively during the second trimester of pregnancy.

In another embodiment, the characteristic expression signature indicates upregulation of at least one protein selected from the group consisting of alstrom syndrome protein 1 (Q8TCU4), prolow-density lipoprotein receptor-related protein (Q07954), syndecan-1 (P18827), hypoxia up-regulated protein 1 (Q9Y4L1), dentrix matrix protein 4 (Q81XL6), leucine-rich repeat and calponin homology (Q5VUJ6), plectin-1 (Q15149), and collagen α-2(IX) chain (Q14055).

In yet another embodiment, the characteristic expression signature indicates upregulation of at least two of such proteins.

In a further embodiment, the characteristic expression signature indicates upregulation of at least three of such proteins.

In a still further embodiment, the characteristic expression signature indicates upregulation of all of the listed proteins.

In another aspect, the invention concerns a proteomic profile of healthy maternal serum in the third trimester of pregnancy.

In one embodiment, the characteristic expression signature indicates upregulation of at least one protein selected from the group consisting of pappalysin-1 (Q13219), apolipoprotein C-III (P02656), pregnancy-specific β-1-glycoprotein-1 (P11465), apolipoprotein C-I (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-4(V) chain (P25940), leucyl cystinyl aminopeptidase (Q9UIQ6), collagen α-1(I) chain (Q15201), and macrophage colony-stimulating factor (P07333).

In another embodiment, characteristic expression signature indicates upregulation of at least two of such proteins.

In yet another embodiment, the characteristic expression signature indicates upregulation of at least three of such proteins.

In a further embodiment, the characteristic expression signature indicates upregulation of all of such proteins.

In a further aspect, the invention concerns a method for diagnosing a pathologic maternal or fetal condition comprising comparing the proteomic profile of a serum sample obtained from a pregnant human subject to the proteomic profile of maternal serum during healthy gestation of the same gestational age, and determining that said pathologic maternal or fetal condition is present, if there is at least one characteristic expression signature differentiating between the proteomic profiles compared.

In one embodiment, the serum sample is obtained in the first trimester of pregnancy.

In another embodiment, the characteristic expression signature indicates upregulation of at least one protein selected from the group consisting of pappalysin-1 (Q13219), apolipoprotein C-III (P02656), apolipoprotein A (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), apolipoprotein(a) (P08519), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-1(XVII) chain (Q9UMD9), leucyl-cystinyl aminopeptidase (Q9UIQ6), collagen α-1(I) chain (Q15201), macrophage colony-stimulating factor (P07333), and pregnancy-specific β-1-glycoprotein 2 (P11465).

In a further embodiment, the serum sample is obtained in the second trimester of pregnancy.

In a still further embodiment, the characteristic expression signature indicates upregulation of at least one protein selected from the group consisting alstrom syndrome protein 1 (Q8TCU4), prolow-density lipoprotein receptor-related protein (Q07954), syndecan-1 (P18827), hypoxia up-regulated protein 1 (Q9Y4L1), dentrix matrix protein 4 (Q81XL6), leucine-rich repeat and calponin homology (Q5VUJ6), plectin-1 (Q15149), and collagen α-2(IX) chain (Q14055).

In a different embodiment, the serum sample is obtained in the third trimester of pregnancy.

In another embodiment the characteristic expression signature indicates upregulation of at least one protein selected from the group consisting of pappalysin-1 (Q13219), apolipoprotein C-III (P02656), apolipoprotein A (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), apolipoprotein(a) (P08519), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-1(XVII) chain (Q9UMD9), leucyl-cystinyl aminopeptidase (Q9UIQ6), collagen α-1(I) chain (Q15201), macrophage colony-stimulating factor (P07333), and pregnancy-specific β-1-glycoprotein 2 (P11465).

In another aspect, the instant invention concerns a report comprising the results of and/or diagnosis based on a test comprising comparing the proteomic profile of a serum sample obtained from a pregnant human subject to the proteomic profile of maternal serum during healthy gestation of the same gestational age, and determining that said pathologic maternal or fetal condition is present, if there is at least one characteristic expression signature differentiating between the proteomic profiles compared.

In yet another aspect, the instant invention includes a tangible medium storing the results of and/or diagnosis based on a test comprising comparing the proteomic profile of a serum sample obtained from a pregnant human subject to the proteomic profile of maternal serum during healthy gestation of the same gestational age, and determining that said pathologic maternal or fetal condition is present, if there is at least one characteristic expression signature differentiating between the proteomic profiles compared. In another aspect, the invention concerns a method for determining the state of maternal or fetal health, comprising comparing a proteomic profile of a test sample of maternal serum obtained from a pregnant female mammalian subject with a proteomic profile of normal maternal serum comprising a unique expression signature wherein the unique expression signature comprises information of the expression of proteins which exhibit continuous upregulation from the first trimester to term. In one embodiment, the characteristic expression signature indicates upregulation of two proteins selected from the group consisting of Chorionic somatomammotropin hormone (P01243), Pregnancy-specific beta-1-glycoprotein 1 (P11464), Choriogonadotropin subunit beta (P01233), Pappalysin-1 (Q13219), and Apolipoprotein C-III (P02656). In other embodiments, the characteristic expression signature indicates upregulation of at least three of said proteins. In yet other embodiments, the characteristic expression signature indicates upregulation of at least four of said proteins. In still yet other embodiments, the characteristic expression signature indicates upregulation of all of said proteins.

In one embodiment, the subject is a human patient.

In certain embodiments, a deviation from the proteomic profile of normal maternal serum indicates risk of a maternal or a fetal condition. In some embodiments, the maternal condition is selected from the group consisting of intrauterine infection, preeclampsia, and preterm labor. In some other embodiments, the fetal condition is selected from the group consisting of chromosomal aneuploidies, congenital malformation, fetal infection, gestational age and fetal maturity. In certain embodiments, the chromosomal aneuploidy is selected from the group consisting of Down syndrome, trisomy-13, trisomy-18, Turner syndrome, and Klinefelter syndrome.

In one embodiment, the determination of the state of maternal or fetal health is made during the first trimester. In another embodiment, the determination of the state of maternal or fetal health is made during the second trimester. In yet another embodiment, the determination of the state of maternal or fetal health is made during the third trimester.

In another aspect, the instant invention includes a report comprising the results of and/or diagnosis based on a test comprising comparing a proteomic profile of a test sample of maternal serum obtained from a pregnant female mammalian subject with a proteomic profile of normal maternal serum comprising a unique expression signature wherein the unique expression signature comprises information of the expression of proteins which exhibit continuous upregulation from the first trimester to term.

In yet another aspect, the instant invention includes a tangible medium storing the results of and/or diagnosis based on a test comprising comparing a proteomic profile of a test sample of maternal serum obtained from a pregnant female mammalian subject with a proteomic profile of normal maternal serum comprising a unique expression signature wherein the unique expression signature comprises information of the expression of proteins which exhibit continuous upregulation from the first trimester to term.

In still another aspect, the invention includes a method for determining the state of maternal or fetal health, comprising comparing a proteomic profile of a test sample of maternal serum obtained from a mammalian subject with a proteomic profile of normal maternal serum comprising a unique expression signature wherein the unique expression signature comprises information of the expression of proteins which exhibit continuous down regulation from the first trimester to term. In one embodiment, the characteristic expression signature indicates down regulation of two proteins selected from the group consisting of histidine-rich glycoprotein (SEQ ID NO:62), C-reactive protein (SEQ ID NO:68), thrombospondin-1 (SEQ ID NO:60), 14-3-3 protein zelta/delta (SEQ ID NO:61), peroxiredoxin-2 (SEQ ID NO:63), profilin-1 (SEQ ID NO:64), L-selectin (SEQ ID NO:65), ficolin-2 (SEQ ID NO:66), and GDH/6PGL endoplasmic bifunctional protein (SEQ ID NO:67). In another embodiment, the characteristic expression signature indicates down regulation of at least three of said proteins. In yet another embodiment, the characteristic expression signature indicates down regulation of at least four of said proteins. In still another embodiment, the characteristic expression signature indicates down regulation of all of said proteins.

In one embodiment, the subject is a human patient.

In certain embodiments, a deviation from the proteomic profile of normal maternal serum indicates risk of a maternal or a fetal condition. In some embodiments, the maternal condition is selected from the group consisting of intrauterine infection, preeclampsia, and preterm labor. In some other embodiments, the fetal condition is selected from the group consisting of chromosomal aneuploidies, congenital malformation, fetal infection, gestational age and fetal maturity. In certain embodiments, the chromosomal aneuploidy is selected from the group consisting of Down syndrome, trisomy-13, trisomy-18, Turner syndrome, and Klinefelter syndrome.

In one embodiment, the determination of the state of maternal or fetal health is made during the first trimester. In another embodiment, the determination of the state of maternal or fetal health is made during the second trimester. In yet another embodiment, the determination of the state of maternal or fetal health is made during the third trimester.

In one aspect, the instant invention also provides a report comprising the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit continuous down regulation from the first trimester to term.

In another aspect, the invention further provides a tangible medium storing the results of and/or diagnosis based on a test comparing a proteomic profile of a test sample of maternal serum obtained from a mammalian subject with a proteomic profile of normal maternal serum comprising a unique expression signature wherein the unique expression signature comprises information of the expression of proteins which exhibit continuous down regulation from the first trimester to term.

In still yet another aspect, the instant invention provides a method for determining the state of maternal or fetal health, comprising comparing a proteomic profile of a test sample of maternal serum obtained from a mammalian subject with a proteomic profile of normal maternal serum comprising a unique expression signature wherein the unique expression signature comprises information of the expression of proteins which exhibit upregulation from the first trimester to second trimester followed by a slow down until term. In one embodiment, the characteristic expression signature indicates upregulation from the first trimester to second trimester followed by a slow down until term of at least one protein selected from the group consisting of pregnancy zone protein (SEQ ID NO: 18), corticosteroid-binding globulin (SEQ ID NO:27), and bone-marrow proteoglycan 2 (SEQ ID NO:16). In another embodiment, the characteristic expression signature indicates upregulation from the first trimester to second trimester followed by a slow down until term of at least two of said proteins. In yet another embodiment, the characteristic expression signature indicates upregulation from the first trimester to second trimester followed by a slow down until term of all of said proteins.

In one embodiment, the subject is a human patient.

In certain embodiments, a deviation from the proteomic profile of normal maternal serum indicates risk of a maternal or a fetal condition. In some embodiments, the maternal condition is selected from the group consisting of intrauterine infection, preeclampsia, and preterm labor. In some other embodiments, the fetal condition is selected from the group consisting of chromosomal aneuploidies, congenital malformation, fetal infection, gestational age and fetal maturity. In certain embodiments, the chromosomal aneuploidy is selected from the group consisting of Down syndrome, trisomy-13, trisomy-18, Turner syndrome, and Klinefelter syndrome.

In one embodiment, the determination of the state of maternal or fetal health is made during the first trimester. In another embodiment, the determination of the state of maternal or fetal health is made during the second trimester. In yet another embodiment, the determination of the state of maternal or fetal health is made during the third trimester.

In another aspect, the instant invention provides a report comprising the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit upregulation from the first trimester to second trimester followed by a slow down until term.

In another aspect, the invention provides a tangible medium storing the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit upregulation from the first trimester to second trimester followed by a slow down until term.

In yet another aspect, the invention provides a method for determining the state of maternal or fetal health, comprising comparing a proteomic profile of a test sample of maternal serum obtained from a mammalian subject with a proteomic profile of normal maternal serum comprising a unique expression signature wherein the unique expression signature comprises information of the expression of proteins which exhibit down regulation from the first trimester to second trimester followed by a slow down until term. In one embodiment, the characteristic expression signature indicates down regulation from the first trimester to second trimester followed by a slow down until term of human choriogonadotropin subunit β (SEQ ID NO:29).

In one embodiment, the subject is a human patient.

In certain embodiments, a deviation from the proteomic profile of normal maternal serum indicates risk of a maternal or a fetal condition. In some embodiments, the maternal condition is selected from the group consisting of intrauterine infection, preeclampsia, and preterm labor. In some other embodiments, the fetal condition is selected from the group consisting of chromosomal aneuploidies, congenital malformation, fetal infection, gestational age and fetal maturity. In certain embodiments, the chromosomal aneuploidy is selected from the group consisting of Down syndrome, trisomy-13, trisomy-18, Turner syndrome, and Klinefelter syndrome.

In one embodiment, the determination of the state of maternal or fetal health is made during the first trimester. In another embodiment, the determination of the state of maternal or fetal health is made during the second trimester. In yet another embodiment, the determination of the state of maternal or fetal health is made during the third trimester.

In one aspect, the invention also provides a report comprising the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit down regulation from the first trimester to second trimester followed by a slow down until term.

In another aspect, the invention provides a tangible medium storing the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit down regulation from the first trimester to second trimester followed by a slow down until term.

In yet another aspect, the invention provides, an immunoassay kit comprising antibodies and reagents for the detection of two or more proteins selected from the group consisting of chorionic somatomammotropin hormone (P01243), Pregnancy-specific beta-1-glycoprotein 1 (P11464), Choriogonadotropin subunit beta (P01233), Pappalysin-1 (Q13219), and Apolipoprotein C-III (P02656).

In still another aspect, the invention provides a proteomic profile of healthy maternal serum from a pregnant subject, wherein the pregnancy resulted from in vitro fertilization.

In another aspect, the invention provides a method for determining the state of placental health, comprising comparing a proteomic profile of a test sample of maternal serum obtained from a mammalian subject whose pregnancy resulted from in vitro fertilization with the proteomic profile of a normal sample.

In yet another aspect, the invention provides a method for predicting small for gestational age comprising comparing the proteomic profile of a serum sample obtained from a pregnant human subject to the proteomic profile of maternal serum during healthy gestation of the same gestational age, and determining that said small for gestational age is more likely than not to be present, if there is at least one characteristic expression signature differentiating between the proteomic profiles compared.

In still yet another aspect, the invention provides a method for predicting fetal loss comprising comparing the proteomic profile of a serum sample obtained from a pregnant human subject to the proteomic profile of maternal serum during healthy gestation of the same gestational age, and determining that said fetal loss is more likely than not to occur, if there is at least one characteristic expression signature differentiating between the proteomic profiles compared.

In one embodiment of the claimed methods, the comparison of proteomic profiles is implemented using an apparatus adapted to determine the expression of said proteins. In certain embodiments, the comparison is performed by using a software program executed by a suitable processor. In some embodiments, program is embodied in software stored on a tangible medium. In certain embodiments, the tangible medium is selected from the group consisting of a flash drive, a CD-ROM, a floppy disk, a hard drive, a DVD, and a memory associated with the processor.

In other embodiments, the claimed methods further comprise the step of preparing a report recording the results of said comparison or the diagnosis. In certain embodiments, the report is recorded or stored on a tangible medium. In some embodiments, the tangible medium is paper. In other embodiments, the tangible medium is selected from the group consisting of a flash drive, a CD-ROM, a floppy disk, a hard drive, a DVD, and a memory associated with the processor.

In yet other embodiments, the claimed methods further comprise the step of communicating the results of said diagnosis to an interested party. In certain embodiments, the interested party is the patient or the attending physician. In some embodiments, the communication is in writing, by email, or by telephone.

In another aspect, the invention also provides an immunoassay kit comprising antibodies and reagents for the detection of any one or more of the proteins disclosed herein, in any combination.

In yet another aspect, the invention also provides the use of proteins in the preparation or manufacture of proteomic profiles of maternal serum as a means for the early determination of the state of a maternal or fetal condition.

These and further aspects and embodiments of the invention will be apparent from the following description.

### BRIEF DESCRIPTION OF THE DRAWINGS

FIG. 1 depicts functional annotation of maternal serum proteome. Serum proteins are annotated using gene ontology (GO) annotations from NCBI database. * Proteins with no particular functions are marked accordingly. Metabolic, catalytic, and defense response molecules emerged as major components of maternal serum. Complement and coagulation cascades along with pregnancy associated proteins also contributed very well to the overall composition of maternal serum.

FIG. 2 depicts the percent overlap between maternal serum, Human Proteome Organisation (HUPO) plasma and amniotic fluid proteome. Maternal serum proteins found in this study were cross-referenced with HUPO plasma and amniotic fluid (AF) proteome and the corresponding percent proteome overlap is shown (see Example 1). The majority of the maternal serum proteins found in this study were confirmed by other proteomes.

FIG. 3 depicts gestational-age dependent maternal serum protein expression changes. MS/MS spectral counts of maternal serum proteins from all trimesters were mean normalized. (A) The cluster analysis (GeneMaths) provides an overall comparison of the protein expression between 1^{st}, 2^{nd}, and 3^{rd} trimesters. The color scale green to red indicates quantification of protein expression: green denoting the least and red denoting the greatest degree of protein expression. Sub-selected clusters with proteins that were exclusively up regulated during 1^{st} trimester, 2^{nd} trimester, and 3^{rd} trimester are shown in FIG. 3B, FIG. 3C, and FIG. 3D, respectively.

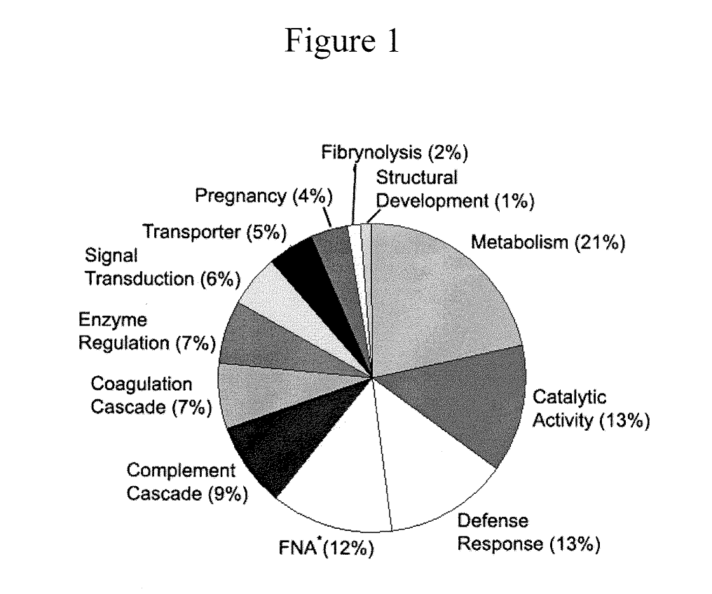

### DETAILED DESCRIPTION OF THE PREFERRED EMBODIMENT

### I. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994) provides one skilled in the art with a general guide to many of the terms used in the present application.

The term “proteome” is used herein to describe a significant portion of proteins in a biological sample at a given time. The concept of proteome is fundamentally different from the genome. While the genome is virtually static, the proteome continually changes in response to internal and external events.

The term “proteomic profile” is used to refer to a representation of the expression pattern of a plurality of proteins in a biological sample, e.g. a biological fluid at a given time. The proteomic profile can, for example, be represented as a mass spectrum, but other representations based on any physicochemical or biochemical properties of the proteins are also included. Thus the proteomic profile may, for example, be based on differences in the electrophoretic properties of proteins, as determined by two-dimensional gel electrophoresis, e.g. by 2-D PAGE, and can be represented, e.g. as a plurality of spots in a two-dimensional electrophoresis gel. Differential expression profiles may have important diagnostic value, even in the absence of specifically identified proteins. Single protein spots can then be detected, for example, by immunoblotting, multiple spots or proteins using protein microarrays. The proteomic profile typically represents or contains information that could range from a few peaks to a complex profile representing 50 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50, or at least 60, or at least 65, or at least 70, or at least 75, or at least 80, or at least 85, or at least 85, or at least 90, or at least 95, or at least 100, or at least 125, or at least 150, or at least 175, or at least 200 proteins.

The term “pathologic condition” is used in the broadest sense and covers all changes and phenomena that compromise the well-being of a subject. Pathologic maternal conditions include, without limitation, intra-amniotic infection, conditions of fetal or maternal origin, such as, for example preeclampsia, and preterm labor and delivery. Pathologic fetal conditions include, without limitation, chromosomal defects (aneuploidies), such as Down syndrome, and all abnormalities in gestational age and fetal maturity.

The term “state of a pathologic [maternal or fetal] condition” is used herein in the broadest sense and refers to the absence, presence, extent, stage, nature, progression or regression of the pathologic condition.

The term “unique expression signature” is used to describe a unique feature or motif within the proteomic profile of a biological sample (e.g. a reference sample) that differs from the proteomic profile of a corresponding normal biological sample (obtained from the same type of source, e.g. biological fluid) in a statistically significant manner.

By “small for gestational age (SGA)” is meant a fetus whose birth weight is a weight less than 2,500 gm (5 lbs. 8 oz.) or below the 10^{th} percentile for gestational age according to U.S. tables of birth weight for gestational age by race, parity, and infant sex as defined by World Health Organization (WHO) (Zhang and Bowes, Obstet. Gynecol. 86:200-208, 1995).

The terms “intra-amniotic infection (IAI),” “amniotic fluid infection,” “amnionitis,” and “clinical chorioamnionitis” are used interchangeably, and refer to an acute infection, including, but not restricted to bacterial, of the amniotic fluid and intrauterine contents during pregnancy.

The term “biological fluid” as used herein refers to refers to liquid material derived from a human or other animal. Biological fluids include, but are not limited to, cord blood, neonatal serum, cerebrospinal fluid (CSF), cervical-vaginal fluid (CVF), amniotic fluid, serum, plasma, urine, cerebrospinal fluid, breast milk, mucus, saliva, and sweat.

“Patient response” can be assessed using any endpoint indicating a benefit to the patient, including, without limitation, (1) inhibition, at least to some extent, of the progression of a pathologic condition, (2) prevention of the pathologic condition, (3) relief, at least to some extent, of one or more symptoms associated with the pathologic condition; (4) increase in the length of survival following treatment; and/or (5) decreased mortality at a given point of time following treatment.

The term “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Congenital malformation” is an abnormality which is non-hereditary but which exists at birth.

The designation of any particular protein, as used herein, includes all fragments, precursors, and naturally occurring variants, such as alternatively spliced and allelic variants and isoforms, as well as soluble forms of the protein named, along with native sequence homologs (including all naturally occurring variants) in other species. Thus, for example, when it is stated that the abundance of haptoglobin precursor (Swiss-Prot Acc. No. P00738) is tested, the statement specifically includes testing any fragments, precursors, or naturally occurring variant of the protein listed under Swiss-Prot Acc. No. P00738, as well as its non-human homologs and naturally occurring variants thereof, if subject is non-human.

### II. Detailed Description

The present invention concerns a global maternal serum proteome map and its changes during healthy gestation. Accordingly, the present invention provides an important tool for plasma-based maternal-fetal diagnostics. In another aspect, the invention concerns the use of proteins in the preparation or manufacture of proteomic profiles as a means for the early determination of the state of a maternal or fetal condition. The invention utilizes proteomics techniques well known in the art, as described, for example, in the following textbooks, the contents of which are hereby expressly incorporated by reference: Proteome Research: New Frontiers in Functional Genomics (Principles and Practice), M. R. Wilkins et al., eds., Springer Verlag, 1007; 2-D Proteome Analysis Protocols, Andrew L Link, editor, Humana Press, 1999; Proteome Research: Two-Dimensional Gel Electrophoresis and Identification Methods (Principles and Practice), T. Rabilloud editor, Springer Verlag, 2000; Proteome Research Mass Spectrometry (Principles and Practice), P. James editor, Springer Verlag, 2001; Introduction to Proteomics, D. C. Liebler editor, Humana Press, 2002; Proteomics in Practice: A Laboratory Manual of Proteome Analysis, R. Westermeier et al., eds., John Wiley & Sons, 2002.

One skilled in the art will recognize many methods and materials similar or equivalent to those described herein, which could be used in the practice of the present invention. Indeed, the present invention is in no way limited to the methods and materials described.

1. Identification of Proteins and Polypeptides Expressed in Biological Fluids

According to the present invention, proteomics analysis of biological fluids can be performed using a variety of methods known in the art. Biological fluids include, for example, cervical-vaginal fluid (CVF), amniotic fluid, serum, plasma, urine, cerebrospinal fluid, breast milk, mucus, and saliva.

Typically, protein patterns (proteome maps) of samples from different sources, such as normal biological fluid (normal sample) and a test biological fluid (test sample), are compared to detect proteins that are up- or down-regulated in a disease. These proteins can then be excised for identification and full characterization, e.g. using peptide-mass fingerprinting and/or mass spectrometry and sequencing methods, or the normal and/or disease-specific proteome map can be used directly for the diagnosis of the disease of interest, or to confirm the presence or absence of the disease.

In comparative analysis, it is important to treat the normal and test samples exactly the same way, in order to correctly represent the relative abundance of proteins, and obtain accurate results. The required amount of total proteins will depend on the analytical technique used, and can be readily determined by one skilled in the art. The proteins present in the biological samples are typically separated by two-dimensional gel electrophoresis (2-DE) according to their pI and molecular weight. The proteins are first separated by their charge using isoelectric focusing (one-dimensional gel electrophoresis). This step can, for example, be carried out using immobilized pH-gradient (IPG) strips, which are commercially available. The second dimension is a normal SDS-PAGE analysis, where the focused IPG strip is used as the sample. After 2-DE separation, proteins can be visualized with conventional dyes, like Coomassie Blue or silver staining, and imaged using known techniques and equipment, such as, e.g. Bio-Rad GS800 densitometer and PDQUEST software, both of which are commercially available. Individual spots are then cut from the gel, destained, and subjected to tryptic digestion. The peptide mixtures can be analyzed by mass spectrometry (MS). Alternatively, the peptides can be separated, for example by capillary high pressure liquid chromatography (HPLC) and can be analyzed by MS either individually, or in pools.

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. Mass spectrometry has been widely used in protein analysis, especially since the invention of matrix-assisted laser-desorption ionisation/time-of-flight (MALDI-TOF) and electrospray ionisation (ESI) methods. There are several versions of mass analyzer, including, for example, MALDI-TOF and triple or quadrupole-TOF, or ion trap mass analyzer coupled to ESI. Thus, for example, a Q-T of-2 mass spectrometer utilizes an orthogonal time-of-flight analyzer that allows the simultaneous detection of ions across the full mass spectrum range. For further details see, e.g. Chemusevich et al., J. Mass Spectrom. 36:849-865 (2001).

If desired, the amino acid sequences of the peptide fragments and eventually the proteins from which they derived can be determined by techniques known in the art, such as certain variations of mass spectrometry, or Edman degradation.

2. Early Detection of Pre-Eclampsia

Preeclampsia, defined as maternal hypertension accompanied by proteinuria, edema, or both, occurs in 7% of pregnancies not terminating in the first trimester. Although the cause is unknown, it is more common in extremes of age in childbearing, maternal diabetes, pregnancies with multiple gestations, and pre-existing maternal renal disease and or hypertension. Preeclampsia is associated with increases in perinatal mortality, and may also lead to eclampsia, characterized by maternal seizures and increased maternal mortality.

Complications of preeclampsia include intrauterine growth retardation (IUGR), small for gestational age (SGA) and HELLP syndrome. Small for Gestational Age (SGA) babies are those whose birth weight lies below the 10^{th} percentile for that gestational age (see above). The incidence of SGA in developed countries is 8.1%. Pre-eclampsia is a condition known to be associated with intrauterine fetal growth restriction (IUGR) and SGA. The etiology, however, can be maternal, fetal or placental. Fetal risk factors include, for example, chromosomal abnormality and infection. Maternal risk factors include, for example, preeclampsia, thrombophilias, antiphospholipid syndrome, defective placentation, sickle cell anemia, drug use, alcohol, and smoking. Accurate diagnosis is complicated by ultra sound assessments and accurate estimation of gestational age. Development of early and reliable markers for SGA is imperative to allow for therapy and intervention to optimize the outcome for the neonate and mother.

HELLP, a syndrome consisting of Hemolysis, Elevated liver enzyme Levels and Low Platelet count, is an obstetric complication that is frequently misdiagnosed at initial presentation. HELLP syndrome occurs in approximately 0.2 to 0.6 percent of all pregnancies. The mainstay of therapy is supportive management, including seizure prophylaxis and blood pressure control in patients with hypertension. Because the symptoms of HELLP syndrome are variable, diagnosis is often delayed. Early diagnosis, however, is critical, and thus, development of early and reliable markers for HELLP syndrome is imperative to allow for therapy and intervention to optimize the outcome for the neonate and mother.

Currently the mainstay of therapy for preeclampsia is delivery and anticonvulsant prophylaxis with magnesium sulfate. Prior to the advent of magnesium sulfate therapy, the observed maternal mortality was 20-30%. However, with prompt diagnosis, allowing anticonvulsant therapy with magnesium sulfate, anti-hypertensives, and delivery the maternal mortality has been reduced to near zero.

Unfortunately, the diagnosis of preeclampsia based upon commonly recognized symptoms and signs is frequently difficult, and occurs late in the course of the disease. Frequently fetal compromise in growth or well-being is the first recognized manifestation of preeclampsia. Laboratory markers for preeclampsia include quantitation of proteinuria, and elevated serum concentrations of uric acid or creatinine. There are no currently available serum markers for early preeclampsia or markers which identify women which will develop preeclampsia. Recently prospective serum markers including leptin and uric acid have been associated with subsequent preeclampsia in one study (Gursoy T, et al. Preeclampsia disrupts the normal physiology of leptin.: Am J Perinatol. 19(6):303-10, 2002) but much work is needed to confirm these findings. Development of early and reliable markers for preeclampsia is imperative to allow for therapy and intervention to optimize the outcome for the neonate and mother.

3. Detection and Diagnosis of Maternal/Fetal Conditions Using a Global Maternal Serum Proteome Map and its Changes

As noted before, in the context of the present invention the term “proteomic profile” is used to refer to a representation of the expression pattern of a plurality of proteins in a biological sample, e.g. maternal serum at a given time. The proteomic profile can, for example, be represented as a mass spectrum, but other representations based on any physicochemical or biochemical properties of the proteins are also included. Diagnosis of a particular disease can be based on characteristic differences (unique expression signatures) between a normal proteomic profile, and proteomic profile of the same biological fluid obtained under the same circumstances, when the disease or pathologic condition to be diagnosed is present. The unique expression signature can be any unique feature or motif within the proteomic profile of a test or reference biological sample that differs from the proteomic profile of a corresponding normal biological sample obtained from the same type of source, in a statistically significant manner. For example, if the proteomic profile is presented in the form of a mass spectrum, the unique expression signature is typically a peak or a combination of peaks that differ, qualitatively or quantitatively, from the mass spectrum of a corresponding normal sample. Thus, the appearance of a new peak or a combination of new peaks in the mass spectrum, or any statistically significant change in the amplitude or shape of an existing peak or combination of existing peaks, or the disappearance of an existing peak, in the mass spectrum can be considered a unique expression signature.

A particular pathologic maternal/fetal condition can be diagnosed by comparing the proteomic profile of a biological fluid, such as maternal serum, obtained from the subject to be diagnosed with the proteomic profile of a normal biological fluid of the same kind, obtained and treated the same manner. If the proteomic profile of the test sample is essentially the same as the proteomic profile of the normal sample, the subject is considered to be free of the subject pathologic maternal/fetal condition. If the proteomic profile of the test sample shows a unique expression signature relative to the proteomic profile of the normal sample, the subject is diagnosed with the maternal/fetal condition in question.

In the methods of the present invention the proteomic profile of a normal biological sample plays an important diagnostic role. As discussed above, if the proteomic profile of the test sample is essentially the same as the proteomic profile of the normal biological sample, the patient is diagnosed as being free of the pathologic maternal/fetal condition to be identified. This “negative” diagnosis is of great significance, since it eliminates the need of subjecting a patient to unnecessary treatment or intervention, which could have potential side-effects, or may otherwise put the patient, fetus, or neonate at risk. The data are analyzed to determine if the differences are statistically significant.

The sensitivity of the diagnostic methods of the present invention can be enhanced by removing the proteins found both in normal and diseased proteome at essentially the same expression levels (common proteins, such as albumin and immunoglobulins) prior to analysis using conventional protein separation methods. The removal of such common proteins, which are not part of the unique expression signature, results in improved sensitivity and diagnostic accuracy. Alternatively or in addition, the expression signatures of the common proteins can be eliminated (or signals can be removed) during computerized analysis of the results, typically using spectral select algorithms, that are machine oriented, to make diagnostic calls.

Statistical methods for comparing proteomic profiles are well known in the art. For example, in the case of a mass spectrum, the proteomic profile is defined by the peak amplitude values at key mass/charge (M/Z) positions along the horizontal axis of the spectrum. Accordingly, a characteristic proteomic profile can, for example, be characterized by the pattern formed by the combination of spectral amplitudes at given M/Z vales. The presence or absence of a characteristic expression signature, or the substantial identity of two profiles can be determined by matching the proteomic profile (pattern) of a test sample with the proteomic profile (pattern) of a reference or normal sample, with an appropriate algorithm. A statistical method for analyzing proteomic patterns is disclosed, for example, in Petricoin III, et al., The Lancet 359:572-77 (2002).; Issaq et al., Biochem Biophys Commun 292:587-92 (2002); Ball et al., Bioinformatics 18:395-404 (2002); and Li et al., Clinical Chemistry Journal, 48:1296-1304 (2002).

In a particular embodiment, the diagnostic tests of the present invention are performed in the form of protein arrays or immunoassays.

4. Protein Arrays

In recent years, protein arrays have gained wide recognition as a powerful means to detect proteins, monitor their expression levels, and investigate protein interactions and functions. They enable high-throughput protein analysis, when large numbers of determinations can be performed simultaneously, using automated means. In the microarray or chip format, that was originally developed for DNA arrays, such determinations can be carried out with minimum use of materials while generating large amounts of data.

Although proteome analysis by 2D gel electrophoresis and mass spectrometry, as described above, is very effective, it does not always provide the needed high sensitivity and this might miss many proteins that are expressed at low abundance. Protein microarrays, in addition to their high efficiency, provide improved sensitivity.

Protein arrays are formed by immobilizing proteins on a solid surface, such as glass, silicon, micro-wells, nitrocellulose, PVDF membranes, and microbeads, using a variety of covalent and non-covalent attachment chemistries well known in the art. The solid support should be chemically stable before and after the coupling procedure, allow good spot morphology, display minimal nonspecific binding, should not contribute a background in detection systems, and should be compatible with different detection systems.

In general, protein microarrays use the same detection methods commonly used for the reading of DNA arrays. Similarly, the same instrumentation as used for reading DNA microarrays is applicable to protein arrays.

Thus, capture arrays (e.g. antibody arrays) can be probed with fluorescently labelled proteins from two different sources, such as normal and diseased biological fluids. In this case, the readout is based on the change in the fluorescent signal as a reflection of changes in the expression level of a target protein. Alternative readouts include, without limitation, fluorescence resonance energy transfer, surface plasmon resonance, rolling circle DNA amplification, mass spectrometry, resonance light scattering, and atomic force microscopy.

For further details, see, for example, Zhou H, et al., Trends Biotechnol. 19:S34-9 (2001); Zhu et al., Current Opin. Chem. Biol. 5:40-45-(2001); Wilson and Nock, Angew Chem Int Ed Engl 42:494-500 (2003); and Schweitzer and Kingsmore, Curr Opin Biotechnol 13:14-9 (2002). Biomolecule arrays are also disclosed in U.S. Pat. No. 6,406,921, issued Jun. 18, 2002, the entire disclosure of which is hereby expressly incorporated by reference.

5. Immunoassays

The diagnostic assays of the present invention can also be performed in the form of various immunoassay formats, which are well known in the art. There are two main types of immunoassays, homogenous and heterogenous. In homogenous immunoassays, both the immunological reaction between an antigen and an antibody and the detection are carried out in a homogenous reaction. Heterogenous immunoassays include at least one separation step, which allows the differentiation of reaction products from unreacted reagents.

ELISA is a heterogenous immunoassay, which has been widely used in laboratory practice since the early 1970's. The assay can be used to detect antigensin various formats.

In the “sandwich” format the antigen being assayed is held between two different antibodies. In this method, a solid surface is first coated with a solid phase antibody. The test sample, containing the antigen (i.e. a diagnostic protein), or a composition containing the antigen, being measured, is then added and the antigen is allowed to react with the bound antibody. Any unbound antigen is washed away. A known amount of enzyme-labelled antibody is then allowed to react with the bound antigen. Any excess unbound enzyme-linked antibody is washed away after the reaction. The substrate for the enzyme used in the assay is then added and the reaction between the substrate and the enzyme produces a colour change. The amount of visual colour change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labelled antigen and both compete for binding to an anti-antigen antibody attached to a solid surface. Excess free enzyme-labelled antigen is washed off before the substrate for the enzyme is added. The amount of color intensity resulting from the enzyme-substrate interaction is a measure of the amount of antigen in the sample tested.

Homogenous immunoassays include, for example, the Enzyme Multiplied Immunoassay Technique (EMIT), which typically includes a biological sample comprising the compound or compounds to be measured, enzyme-labeled molecules of the compound(s) to be measured, specific antibody or antibodies binding the compound(s) to be measured, and a specific enzyme chromogenic substrate. In a typical EMIT excess of specific antibodies is added to a biological sample. If the biological sample contains the proteins to be detected, such proteins bind to the antibodies. A measured amount of the corresponding enzyme-labelled proteins is then added to the mixture. Antibody binding sites not occupied by molecules of the protein in the sample are occupied with molecules of the added enzyme-labelled protein. As a result, enzyme activity is reduced because only free enzyme-labelled protein can act on the substrate. The amount of substrate converted from a colourless to a coloured form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the Ag-enzyme complex is Ab-bound makes the EMIT a unique system, enabling the test to be performed without a separation of bound from unbound compounds as is necessary with other immunoassay methods.

Part of this invention is also an immunoassay kit. In one aspect, the invention includes a sandwich immunoassay kit comprising a capture antibody and a detector antibody. The capture antibody and detector antibody can be monoclonal or polyclonal. In another aspect, the invention includes a diagnostic kit comprising lateral flow devices, such as immunochromatographic strip (ICS) tests, using immunoflowchromatography. The lateral flow devices employ lateral flow assay techniques as generally described in U.S. Pat. Nos. 4,943,522; 4,861,711; 4,857,453; 4,855,240; 4,775,636; 4,703,017; 4,361,537; 4,235,601; 4,168,146; 4,094,647, the entire contents of each of which is incorporated by reference. In yet another aspect, the immunoassay kit may comprise, for example, in separate containers (a) monoclonal antibodies having binding specificity for the polypeptides used in the diagnosis of a particular maternal/fetal condition, such as preeclampsia; (b) and anti-antibody immunoglobulins. This immunoassay kit may be utilized for the practice of the various methods provided herein. The monoclonal antibodies and the anti-antibody immunoglobulins may be provided in an amount of about 0.001 mg to about 100 grams, and more preferably about 0.01 mg to about 1 gram. The anti-antibody immunoglobulin may be a polyclonal immunoglobulin, protein A or protein G or functional fragments thereof, which may be labeled prior to use by methods known in the art. The diagnostic kit may further include where necessary agents for reducing background interference in a test, agents for increasing signal, software and algorithms for combining and interpolating marker values to produce a prediction of clinical outcome of interest, apparatus for conducting a test, calibration curves and charts, standardization curves and charts, and the like. The test kit may be packaged in any suitable manner, typically with all elements in a single container along with a sheet of printed instructions for carrying out the test.

6. Diagnostic and Treatment Methods

The diagnostic methods of the present invention are valuable tools for practicing physicians to make quick treatment decisions, which are often critical for the survival of the infant and/or mother. Thus, for example, if a pregnant woman shows symptoms of a maternal/fetal condition, it is important to take immediate steps to treat the condition and improve the chances of the survival of the fetus and limit the risks to the mother's health.

Following the measurement or obtainment of the expression levels of the proteins identified herein, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the expression level in a test subject of one or more of the biomarkers herein is communicated to the subject as soon as possible after the assay is completed and the diagnosis and/or prediction is generated. The one or more biomarkers identified and quantified in the methods described herein can be contained in one or more panels. The number of biomarkers comprising a panel can include 1 biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, 20 biomarkers, etc. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

To facilitate diagnosis, the reference and/or subject biomarker profiles or expression level of one or more of the biomarkers presented herein of the present invention can be displayed on a display device, contained electronically, or in a machine-readable medium, such as but not limited to, analog tapes like those readable by a VCR, CD-ROM, DVD-ROM, USB flash media, e.g., flash drive, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

Further details of the invention will be apparent from the following non-limiting examples. All references cited throughout the disclosure, and the references cited therein, are expressly incorporated by reference herein.

7. Maternal-Fetal Conditions Benefiting from Early and Non-Invasive Diagnosis

Intra-Amniotic Infection

Intra-amniotic infection (IAI) is an acute bacterial infection of the amniotic fluid and intrauterine contents during pregnancy. Prospective studies indicate that IAI occurs in 4% to 10% of all deliveries (Newton, E. R., Prihoda, T. J., and Gibbs, R. S.: Logistic regression analysis of risk factors for intra-amniotic infection. Obstet. Gynecol. 73:571, 1989; Soper, D. E., Mayhall, C. G., and Dalton, H. P.: Risk factors for intraamniotic infection: a prospective epidemicologic study. American Journal of Obstetrics and Gynecology 161:562, 1989; and Lopez-Zeno, J. A., Peaceman, A. M., Adashek, J. A., and Socol, M. L.: A controlled trial of a program for the active management of labor. N. Engl. J. Med. 326:450, 1992). Other terms used to describe IAI include amniotic fluid infection, amnionitis, and clinical chorioamnionitis. Intra-amniotic infection is clinically diagnosed by maternal fever, uterine tenderness, leukocytosis, and fetal tachycardia and should be distinguished from histologic chorioamnionitis. Intra-amniotic infection is an important cause of maternal and neonatal morbidity. Intra-amniotic infection accounts for 10-40% of cases of febrile morbidity in the peripartum period and is associated with 20-40% of cases of early neonatal sepsis and pneumonia (Newton, E. R.: Chorioamnionitis and intraamniotic infection. Clin. Obstet. Gynecol. 36:795, 1993). Maternal bacteremia occurs in 2-6% of patients with IAI and postpartum infectious morbidity is increased. There is also an increased risk of dysfunctional labor and cesarean delivery among patients with IAI. Duff et al. reported a 75% incidence of dysfunctional labor and a 34% incidence of cesarean delivery among patients who developed intra-amniotic infection while in labor (Duff, P., Sanders, R., and Gibbs, R. S.: The course of labor in term pregnancies with chorioamnionitis. American Journal of Obstetrics and Gynecology 147:391, 1983). Intra-amniotic infection is also associated with increased neonatal morbidity and mortality, particularly among preterm neonates. In general, there is a three to four-fold increase in perinatal mortality among low birth weight neonates born to mothers with IAI (Gibbs, R. S., Castillo, M. A., and Rodgers, P. J.: Management of acute chorioamnionitis. American Journal of Obstetrics and Gynecology 136:709, 1980; Gilstrap, L. C., III, Leveno, K. J., Cox, S. M., Burris, J. S., Mashburn, M., and Rosenfeld, C. R.: Intrapartum treatment of acute chorioamnionitis: impact on neonatal sepsis. Am. J. Obstet. Gynecol. 159:579, 1988). There are also increases in respiratory distress syndrome, intraventricular hemorrhage, and neonatal sepsis Morales, W. J.: The effect of chorioamnionitis on the developmental outcome of preterm infants at one year. Obstetrics and Gynecology 70:183, 1987). Recently, IAI has been implicated in neonatal periventricular leukomalacia and cerebral palsy; the risks of cerebral white matter damage and cerebral palsy are nine-fold greater in the setting of intra-amniotic infection Bejar, R., Wozniak, P., Allard, M., Benirschke, K., Vaucher, Y., Coen, R., Berry, C., Schragg, P., Villegas, I., and Resnik, R.: Antenatal origin of neurologic damage in newborn infants. I. Preterm infants. Am. J. Obstet. Gynecol. 159:357, 1988; Grether, J . K. and Nelson, K. B.: Maternal infection and cerebral palsy in infants of normal birth weight. JAMA 278:207, 1997). Finally, subclinical IAI has been found in at least 10% of women in preterm labor with intact fetal membranes, suggesting that IAI is an important, and potentially preventable, cause of prematurity (Romero, R., Avila, C., Brekus, C. A., and Morotti, R.: The role of systemic and intrauterine infection in preterm parturition. Annuals of the New York Academy of Sciences 622:355, 1991). A literature review by Newton demonstrated incidences of clinical IAI of 41% at gestational ages less than 27 weeks, 15% at gestational ages of 27-37 weeks, and 2% at gestations of 38 weeks or greater (Newton et al., supra). Bacteria indigenous to the lower genital tract have also been recovered from the amniotic fluid of 10-20% of all women in preterm labor with intact fetal membranes without clinical signs of intraamniotic infection (Romero et al., supra), and in up to 67% of women in preterm labor with pregnancies ending at 23-24 weeks (Watts, D. H., Krohn, M. A., Hillier, S. L., and Eschenbach, D. A.: The association of occult amniotic fluid infection with gestational age and neonatal outcome among women in preterm labor. Obstet Gynecol 79:351, 1992). Most of these patients deliver rapidly, and clinically apparent IAI develops in many. These observations support the hypothesis that ascending, initially subclinical intrauterine infections precede preterm labor and may be an important cause of extreme preterm deliveries.

Preeclampsia

Preeclampsia, defined as maternal hypertension accompanied by proteinuria, edema, or both, occurs in 7% of pregnancies not terminating in the first trimester. Although the cause is unknown, it is more common in extremes of age in childbearing, maternal diabetes, pregnancies with multiple gestations, and pre-existing maternal renal disease and or hypertension. Preeclampsia is associated with increases in perinatal mortality, and may also lead to eclampsia, characterized by maternal seizures and increased maternal mortality. Currently the mainstay of therapy for preeclampsia is delivery and anticonvulsant prophylaxis with magnesium sulfate. Prior to the advent of magnesium sulfate therapy, the observed maternal mortality was 20-30%. However, with prompt diagnosis, allowing anticonvulsant therapy with magnesium sulfate, anti-hypertensives, and delivery the maternal mortality has been reduced to near zero.

Unfortunately, the diagnosis of preeclampsia based upon commonly recognized symptoms and signs is frequently difficult, and occurs late in the course of the disease. Frequently fetal compromise in growth or well-being is the first recognized manifestation of preeclampsia. Laboratory markers for preeclampsia include quantitation of proteinuria, and elevated serum concentrations of uric acid or creatinine. There are no currently available serum markers for early preeclampsia or markers which identify women which will develop preeclampsia. Recently prospective serum markers including leptin and uric acid have been associated with subsequent preeclampsia in one study (Gursoy T, et al. Preeclampsia disrupts the normal physiology of leptin: Am J Perinatol. 19(6):303-10, 2002) but much work is needed to confirm these findings. Development of early and reliable markers for preeclampsia is imperative to allow for therapy and intervention to optimize the outcome for the neonate and mother.

Preterm Labor and Delivery

Preterm delivery is defined as birth prior to the 37^{th} completed week of gestation. The incidence of preterm birth in the United States is 10-11% of all live births, and is increasing despite aggressive treatment of preterm labor. Overall, prematurity and its consequences are responsible for 80% of perinatal deaths not attributable to congenital malformations and add approximately $5 billion annually to the national health care budget. Risk factors for preterm birth include non-white race, young age, low socioeconomic status, maternal weight below 55 kg, nulliparity, 1^{st} trimester bleeding, multiple gestations (Meis P J, Michielutte R, Peters T J, et al. Factors associated with preterm birth in Cardiff, Wales: II. Indicated and spontaneous preterm birth. Am J Obstet Gynecol 173:597-602, 1995).

Unfortunately the prediction of patients at risk for spontaneous preterm birth has been generally disappointing (Creasy R K, Iams J D. Preterm labor and delivery. In Maternal-Fetal Medicine, Creasy R K, Resnik R (eds.). W.B. Saunders Company, Philadelphia, Pa. 4^{th} edition, 1999. Pages 498-531). Previous attempts at defining the population at greatest risk for preterm birth, and thereby potentially benefiting from early intervention have included risk-scoring indices, biochemical detection of cervical fetal fibronectin, ultrasound measurement of cervical length, and home uterine activity monitoring. These programs have been both costly, and have been hampered by the inability to predict with accuracy which patients might benefit from early intervention or prophylaxis. All suffer from poor positive predictive value of approximately 30%, with the majority of patients identified as “at risk” delivering at term. Interventions, including pharmacologic treatment to inhibit uterine contractions, are efficacious, but depend upon the early and reliable diagnosis of preterm labor. Early and reliable markers to identify patients at greatest risk for preterm birth are therefore necessary to reduce the tremendous costs and neonatal mortality and morbidity associated with preterm birth.

Chromosomal Aneuploidies

Chromosomal abnormalities are a frequent cause of perinatal morbidity and mortality. Chromosomal abnormalities occur with an incidence of 1 in 200 live births. The major cause of these abnormalities is chromosomal aneuploidy, an abnormal number of chromosomes inherited from the parents. One of the most frequent chromosomal aneuploidies is trisomy-21 (Down syndrome), which has an occurrence of 1 in 800 livebirths (Hook E B, Hamerton J L: The frequency of chromosome abnormalities detected in consecutive newborn studies: Differences between studies: Results by sex and by severity of phenotypic involvement. In Hook E B, Porter I H (eds): Population Cytogenetics, pp 63-79. New York, Academic Press, 1978). The primary risk factor for trisomy-21 is maternal age greater than 35, but 80% of children with trisomy-21 are born to women younger than 35 years of age. Other common aneuploidic conditions include trisomies 13 and 18, Turner Syndrome and Klinefelter syndrome.

Because 80% of children with trisomy-21 are born to women younger than 35 years of age, prenatal diagnostic screening programs designed on the basis of maternal age alone are inefficient. Prenatal screening programs have therefore been supplemented with maternal serum screening for analytes associated with fetal chromosomal aneuploidy, ultrasound, or a combination of both. Candidate serum markers that have been widely utilized include alpha-fetoprotein (AFP), unconjugated estriol, human choriogonadotrophic hormone (hHCG), and inhibin-A. However, with a screen positive rate of 2-5%, the detection rate for trisomy-21 and other aneuploidies has been disappointing, with detection rates of only 70-86% (Cuckle H. Biochemical screening for Down syndrome. Eur J Obstet Gynecol Reprod Biol. 92(1):97-101, 2000). Further, the rate of true positive tests, i.e., trisomy-21 among those with a screen positive test is only 1-2%, resulting in an overall false positive rate in excess of 98%.

The definitive diagnosis of chromosomal aneuploidies following maternal serum screening and ultrasound requires a mid-trimester genetic amniocentesis. This is an invasive procedure associated with a 0.5% risk of loss of the pregnancy. Further, chromosomal analysis of amniotic fluid cells is a labor-intensive and time consuming procedure, taking up to 2 weeks. Reliable tests are therefore necessary to improve the detection of chromosomal aneuploidies from maternal serum, reduce the unacceptably high false positive rate of maternal screening, and increase the speed and efficiency of diagnosis from amniotic fluid following amniocentesis.

Abnormal Placentation

Assisted reproductive technology (ART), including in vitro fertilization (IVF) and intracytoplasmic sperm injection (ICSI), has grown explosively since its development. In its simplest form, IVF consists of pharmaceutical stimulation of the female's ovaries to produce a large number of follicles. Eggs surgically harvested from these follicles are then mixed in the laboratory with the male's sperm. If fertilization is successful, the embryos are incubated for a short time and then transferred back to the female. If one of these embryos implants in the uterine wall, a successful pregnancy may follow. Placentation, the formation and growth of the placenta inside the uterus, occurs following implantation. It has been shown, however, that singleton pregnancies IVF and ICSI are associated with increased risks of abnormal placentation, pre-eclampsia and preterm birth.

The adverse outcomes associated with IVF emphasize the need to identify distinct differences in the maternal serum proteome profile of early placentation in IVF and normal placentation to provide early, selective treatments. Accordingly, one aspect of the invention provides a method for determining the state of placental health.

Small for Gestational Age

Small for Gestational Age (SGA) babies are those whose birth weight lies below the 10^{th} percentile for that gestational age (see above). The incidence of SGA in developed countries is 8.1%. Pre-eclampsia is a condition known to be associated with intrauterine fetal growth restriction (IUGR) and SGA. The etiology, however, can be maternal, fetal or placental. Fetal risk factors include, for example, chromosomal abnormality and infection. Maternal risk factors include, for example, preeclampsia, thrombophilias, antiphospholipid syndrome, defective placentation, sickle cell anemia, drug use, alcohol, and smoking. Accurate diagnosis is complicated by ultra sound assessments and accurate estimation of gestational age. Development of early and reliable markers for SGA is imperative to allow for therapy and intervention to optimize the outcome for the neonate and mother.

Fetal Loss

According to the 2003 revision of the Procedures for Coding Cause of Fetal Death Under ICD-10, the National Center for Health Statistics defines fetal death as “death prior to the complete expulsion or extraction from its mother of a product of human conception, irrespective of the duration of pregnancy and which is not an induced termination of pregnancy. In 25-60% of all cases, the etiology of fetal demise is unknown. In the cases where a cause is clearly identified, the cause of fetal death can be due to fetal, maternal, or placental pathology. Maternal causes include, for example, prolonged pregnancy (>42 wk), poorly controlled diabetes, advanced maternal age, preeclampsia, eclampsia, infection, hypertension, hemoglobinopathy, Rh disease, uterine rupture, antiphospholipid syndrome, and systemic lupus erythematosus. Fetal causes include multiple gestations, intrauterine growth restriction, congenital abnormality, genetic abnormality, infection (e.g., parvovirus B19, CMV, listeria), and hydrops. Placental causes include cord accident, abruption, premature rupture of membranes, vasa previa, fetomaternal hemorrhage, and placental insufficiency.

As fetal loss is a significant condition of unmet medical need, methods of predicting fetal loss are needed to provide early, selective treatments. Accordingly, one aspect of the invention provides a method for predicting fetal loss based on normal maternal serum profiles.

Further details of the invention will be provided in the following non-limiting Examples.

All references cited throughout the disclosure and the references cited therein are hereby expressly incorporated by reference.

### Example 1

### Identification of the Proteome of Maternal Serum

### Materials and Methods

Sample Collection and Processing (Active PE): A total of 44 healthy human subjects were identified prospectively and given informed consent to participate in the study. Subjects were monitored through out their entire pregnancy and all of them delivered at term without any complications. A total of three serum draws, one per trimester, were taken serially from each subject. The mean gestational age of the women at the time of the first, second and third trimester serum draws are 9.9±1.3, 23.48±1.75, and 35.81±1.79 weeks, respectively. Samples were allowed to clot for 30 min., spun down at 500 g, supernatant was collected and stored in −80° C. until further processing. A total of 15 subjects from the group were randomly selected and their serial draws are pooled together into three samples (one per trimester) according to the time of the draw. The mean gestational age of the pooled serum draws from first, second and third trimester are 9.7±1.3 weeks, 22.0±1.7 weeks, and 33.6±3.01 weeks, respectively. Pooled samples are subjected to immuno-depletion followed by two-dimensional liquid chromatography (2-DLC) tandem mass spectrometry.

Immunodepletion of human serum: Serum samples used for 2-DLC experiments were depleted of 12 most abundant proteins (albumin, IgG, IgA, IgM, α-1-anti-trypsin, transferrin, haptoglobin, α-1-acid glycoprotein, α-2-macroglobulin, fibrinogen, apolipoproteins A-I and A-II) using IgY-12 LC2 proteome partitioning system (Beckman Coulter, Fullerton, Calif.). The low abundance protein fraction was collected, concentrated using 5000 MWCO filters (Millipore, Billerica, Mass.), and buffer exchanged with 10 mM Tris (pH 8.4). Protein concentration was determined using a DC protein assay kit (Bio-Rad, Hercules, Calif.).

2-DLC sample processing: Following protein assay, 1 mg portions of samples were digested with trypsin, and resulting peptides were separated with strong cation exchange (SCX) chromatography {Link, 1999 #69; Washburn, 2001 #28}. Samples were dried and dissolved in 105 μL of digestion buffer containing 0.2 M NH₄HCO₃ and 0.3% Rapigest (Waters, Milford, Mass.) (pH 8.5). Cysteine residues were reduced and alkylated by incubating in 12.5 μL of 0.1 M DTT at 50° C. for 45 min followed by dark room incubation in 7 μL of 0.5 M iodoacetamide for another 30 min. Proteins were digested for 2 h at 37° C. by adding 4 μL of 0.1 M CaCl₂ and sequencing grade trypsin (Trypsin Gold, Promega) at an enzyme to substrate ratio of 33:1. Digestion was stopped by adding 60 μL of 0.2 M HCl and resulting peptides were purified using C18 SepPak Plus cartridges (Waters, Milford, Mass.).

SCX chromatography was performed using a 100×2.1 mm polysulfoethyl A column (The Nest Group, Southborough, Mass.). Mobile phase A contained 10 mM potassium phosphate (pH 3) and 25% acetonitrile (ACN). Mobile phase B was identical except that it contained 350 mM KCl. Following loading and washing in mobile phase A, peptides were eluted using a linear gradient of 0-50% B over 45 min, followed by a linear gradient of 50-100% B over 15 min, followed by a 20 min wash at 100% A. A total of 95 one-minute fractions were collected, dried by vacuum centrifugation, and re-dissolved by shaking in 100 μL of 0.1% TFA. Peptide fractions were desalted using a 96-well spin column, Vydac C18 silica (The Nest Group, Southborough, Mass.). The desalted fractions were consolidated into 31 fractions, evaporated, and dissolved in 20 μL of 5% FA for LC-MS/MS analysis.

LC-MS.Ms analysis: Portions of each fraction were analyzed by LC/MS using an Agilent 1100 series capillary LC system and an LTQ ion trap mass spectrometer (Thermo Electron, San Jose, Calif., USA) with an Ion Max electrospray source fitted with a 34-gauge metal needle kit (ThermoFinnigan, San Jose, Calif.). Samples were applied at 20 μL/min to a trap cartridge, and then switched onto a 0.5×250 mm Zorbax SB-C18 column (Agilent Technologies, Palo Alto, Calif., USA) using mobile phase A containing 0.1% FA. Survey mass spectrometry (MS) scans were alternated with 3 data-dependant MS/MS scans using the dynamic exclusion feature of the control software to increase the number of unique peptides analyzed. Mass spectra files were generated using Bioworks Browser software (version 3.1, ThermoFinnigan, San Jose, Calif.) with m/z range of 400 to 4000 Da, a minimum of 15 ions, and a low TIC threshold of 500. A total of 1,802,623 tandem mass spectra were generated from all LC-MS/MS analyses.

Peptide and protein identification: Tandem mass spectra were searched against a composite protein database containing forward and reversed entries (decoy proteins) of Swiss-Prot (version 52.1) database selected for human subspecies. All searches were performed using X! Tandem {Craig, 2004 #36; Fenyo, 2003 #34} search engine configured to use 1.8 Da and 0.4 Da as parent and fragment ion mass tolerances, respectively. No enzyme was specified while deriving peptide candidates from the database. X! Tandem was also configured to search with a fixed carbamidomethyl modification on cysteine residues and several potential in vivo and in vitro modifications. Peptide identifications from samples were assembled into proteins using probabilistic protein identification algorithms {Nesvizhskii, 2003 #37} implemented in Scaffold software (version 1.6, Proteome Software, Portland, Oreg.).

Peptide and protein identifications in all samples were compiled together to generate a comprehensive maternal serum proteome during gestation. Peptide identifications with at least a probability of 0.8 and without any unknown and unexpected modifications are considered as likely to be present in the sample. Protein identifications with at least either three unique peptide identifications in one sample or two unique peptide identifications in at least two samples are considered to be present in maternal serum. Extraneous proteins (trypsin and keratin) and proteins that are subsets (degenerate) of other proteins were removed from the determined proteome.

Label-free quantitation: The total number of tandem mass spectra matched to a protein (spectral counting) is a label-free, sensitive, and semi-quantitative measure for estimating its abundance in complex mixtures {Old, 2005 #29; Liu, 2004 #39; Zybailov, 2005 #43}. The spectral count difference between two complex samples is used to quantify the relative expression of a protein. {Zybailov, 2006 #44; Gravett, 2007 #45; Pereira, 2007 #40; Nagalla, 2007 #66; Pang, 2002 #65}. In this study, maternal serum proteins with at least three unique peptide identifications in at least one sample were considered for label-free quantitation. Protein entries were further curated before subjecting to label-free quantitation in order to reduce the false positive rate. Shared spectral counts of non-degenerate proteins belonging to same family with significant sequence homology (>50%) were combined into single entry. Shared spectral counts of non-degenerate proteins that did not fit the afore-mentioned criteria were assigned to one of the protein using Occam's razor approach. Spectral counts of all immunoglobulin and pregnancy-specific-β-1-glycoprotein variants were collapsed into single entries. Curated proteins were subjected to independent pair-wise comparisons to determine differentially expressed proteins between first and second, first and third, and second and third trimesters.

Table 1 shows the model tested in pair-wise comparisons using either a 2×2 χ² or fisher exact test. If a total of (W+X) number of spectra matched to a protein and (Y+Z) number of spectra did not match to same protein (i.e. matched to other proteins) in both samples, then the hypothesis was, given the distribution of spectral counts for a protein between two samples, as shown in Table 1, what is the probability that counts are evenly distributed across them? Normalization of spectral counts to account for experimental variability was built into the pair-wise comparison model as shown in Table 1. Proteins with total number of spectral counts ≧5 in both samples are subjected to a χ² test. Proteins that did not fit the afore-mentioned criterion are subjected to fisher exact test. The method was automated using a SAS program (version 9.1) and all proteins were independently tested. A protein was considered as significantly differentially expressed between the samples if the hypothesis has a p-value of ≦0.05 in either the χ² or fisher exact test. The fold expression change (FC) of differentially expressed proteins is quantified using the equation described elsewhere {Old, 2005 #29}.

TABLE 1
Spectral Count χ2 Test Model for Label-Free Quantitation

| | Sample | | |
|---|---|---|---|
| Total Spectra | Sample 1 | Sample 2 | |
| Matched to Protein A | W | X | W + X |
| Not Matched to | Y | Z | Y + Z |
| Protein A | | | |
| | W + Y | X + Z | W + X + Y + Z |

Label-free trend analysis: Curated proteins were also independently subjected to orthogonal polynomial trend test {Hubert, 1973 #114}. Protein relative expression change with the progression of pregnancy (i.e. from first trimester to second trimester to third trimester) was assessed with Poisson regression {Alan, 1990 #62} of its spectral count. Orthogonal polynomial contrasts were used to test for the following trends across all trimesters: linear up regulation, linear down regulation, up regulated from first to second trimesters and down regulated thereafter to term (upward spike), and down regulated from first to second trimesters and up regulated thereafter to term (downward spike). As the trend test statistic is chosen to reflect the anticipated trends, it is more sensitive at detecting them than a normal χ² test. The level of significance for passing the trend test was set at 0.05 and test was automated using a GENMOD procedure in SAS software (version 9.1).

Enzyme-Linked Immunosorbent Assay: Concentrations of selected candidate biomarker proteins in first, second, and third trimester maternal serum samples (n=44) were estimated by enzyme-linked immunosorbent assay (ELISA) {Clark, 1977 #60; Nerurkar, 1984 #61}. Specific antibodies and pure proteins for Pappalysin-1 (PAPP-A), pregnancy-specific-β-1-glycoprotein 1 (PSG1), human chorionic gonadotropin subunit β (βHCG), Apolipoprotein C-III (ApoC-III), and chorionic somatomammotrophin hormone (CSH) were obtained from appropriate sources. To facilitate the detection, the antibodies were conjugated with either biotin using Sulfo-NHS-Biotinylation kit (Pierce Biotechnology Inc., Rockford, Ill.) or HRP. Samples and antibodies were diluted in 1% BSA with reaction volume set at 100 μL/well and all the incubations were performed at room temperature (RT) for 1 h on a shaker at 300 rpm, unless otherwise mentioned. All the washing steps in the ELISA were performed by an automated PW-384 power washer (Tecan, Switzerland) using PBS with 0.5% Tween-20 (PBST).

For the ELISA, Reacti bind 96-well microtiter assay plate (Pierce Biotechnology Inc., Rockford, Ill.) was first coated with 100 μL/well by the purified IgG grade antibody at a concentration of 2.0 μg/ml, prepared in carbonate-bicarbonate buffer, 0.1 M, pH 9.6, and incubated overnight at 4° C. The maximum binding capacity of the individual well was 400 ng/cm². After the overnight incubation, the plate was washed with 650 μL/well of PBST, and blocked with 200 μL of 3% of BSA (prepared in PBS), for 1.5 h at RT. The plates were then washed with 650 μL of PBST. Appropriate dilutions of the pure antigen and the serum samples were prepared in triplicates and incubated for 1 h. The wells were then washed with 1.65 mL of PBST. Plates were incubated with appropriate dilution of biotin- or HRP-conjugated secondary antibody for 1 h, and washed with 1.65 ml of PBST. Appropriate horseradish peroxidase (HRP) conjugate was added (if necessary) at a concentration of 0.1 μg/mL and incubated for 45 min, and washed with 2 ml of PBST. TMB substrate was added and incubated at RT for 5-15 min for the color development. The reaction was halted by adding 100 μL of 2NH₂SO₄ and thus formed yellow color was read at 450 nm on a Spectra max plus microplate reader (Molecular Devices corporation, Sunnyvale, Calif.). A four-parameter standard curve was generated for every ELISA plate by plotting concentrations of the known proteins against their optical density (OD) values using the SoftmaxPro software (version 5.2, Molecular Devices corporation, Sunnyvale, Calif.). The concentrations of the individual proteins were estimated from the average values of triplicates in comparison to the standard curve. The large number of samples used in the study required the use of multiple plates. Hence, a reference standard (known concentration of pure proteins) was spotted on all the plates and the ELISA values from all the plates are normalized with respect to the reference standard in order to correct for plate-to-plate variation.

Statistical analysis of ELISA data: Candidate protein biomarker concentrations (expressed as ng/mL) measured by ELISA experiments in first (n=44), second (n=44) and third (n=44) trimester maternal serum samples were log transformed before subjecting them to statistical analysis. Subjects with adequate overall protein in their samples, but with ELISA values under detectable limit for a particular protein were assigned a value of 0.1 rather than 0 to facilitate log-transformation. Independent pair-wise comparisons of log-transformed protein concentrations between first and second, first and third, and second and third trimester subjects was performed using one-way analysis of variance (ANOVA) test. Observing statistically significant differences in measured protein concentrations between trimester groups does not necessarily mean that the protein has the power to robustly distinguish between them. Therefore, simple logistic regression models {Hosmer, 2000 #56} with protein concentration and sample status were independently fit for each biomarker. The predicted values from these models were used to create Receiver Operating Characteristic (ROC) curves {Pepe, 2003 #57}. ROC curves are plots of the true positive fraction of a test (sensitivity) versus the false positive fraction (1-specificity) across the entire continuum of predicted values. The area under the curve for a given protein should be between 0.5 (poor discriminant) to 1.0 (perfect discriminant), and can be expressed probabilistically as the probability that a randomly selected pair of trimester subjects is correctly classified. Standard errors for the AUROC were conducted based on percentiles of bootstrapped distributions {Pepe, 2003 #57}. The comparative analyses, logistic regression models, and ROC curves were generated using SAS software (version 9.1).

Results

A prospective cohort of 44 human maternal subjects was followed through their entire pregnancy to measure gestational-age dependent changes in maternal serum. Serial serum draws from the subjects were taken during first, second and third trimesters. Serial serum draws of 15 subjects from first, second and third trimesters were subjected to two-dimensional liquid chromatography tandem mass spectrometry (2-DLC/MS/MS). Peptides and proteins from all the experiments are compiled together to develop a comprehensive maternal serum proteome during gestation. Spectral counts of protein identifications were subjected to label-free quantitation to identify gestational-age dependent maternal serum changes. Selected protein biomarkers from label-free quantitation were validated using enzyme-linked immuno assays (ELISA). Protein expression trends in maternal serum during pregnancy were identified using a label-free trend analysis.

A total of 453 proteins with at least two unique confident (probability >=0.8) peptides were identified in this study. Any decoy proteins that also passed the above-described protein identification criteria were considered as false positive identifications. In order to reduce the false positive rate of the protein identifications, proteins with at least either three unique confident peptide identifications in one sample or two unique confident peptide identifications in two samples were considered to be likely present in the maternal plasma. A total of 266 proteins were identified using the above criteria. The total number of MS2 spectra matched to a protein is a semi-quantitative measurement of its abundance in complex mixtures {Liu, 2004 #39}. Proteins were sorted based on decreasing order of their corresponding total spectral counts from all samples. Serum proteins with at least three unique peptide identifications in at least one of the samples were subjected to a 2×2 chi-square test.

TABLE 2
Differentially Expressed Serum Proteins Between 1st, 2nd and 3rd Trimester Pregnancy Controls

| | | Fold Change ^{a} | | | 2 × 2 Chi-square P-values | | | | |
|---|---|---|---|---|---|---|---|---|---|
| Swiss-Prot | | 2nd vs. | 3rd vs. | 3rd vs. | 2nd vs. | 3rd vs. | 3rd vs. | Trend Test ^{b} | |
| Accession | Protein name | 1st | 1st | 2nd | 1st | 1st | 2nd | P-value | Trend ^{c} |
| Q13219 | Pappalysin-1 (SEQ ID NO: 1) | 65.0 | 175.2 | 2.7 | <0.0001 | <0.0001 | <0.0001 | <0.0001 | 1 < 2 < 3 |
| Q9UIQ6 | Leucyl-cystinyl aminopeptidase | 5.2 | 13.3 | 2.6 | 0.0330 | <0.0001 | 0.0170 | <0.0001 | 1 < 2 < 3 |
| | (SEQ ID NO: 2) | | | | | | | | |
| O43184 | ADAM 12 (SEQ ID NO: 3) | 2.1 | 9.3 | 4.5 | | <0.0001 | 0.0003 | 0.0021 | 1 < 2 < 3 |
| P07333 | Macrophage colony-stimulating | 3.7 | 9.2 | 2.5 | | 0.0007 | 0.0770 | <0.0001 | 1 < 2 < 3 |
| | factor 1 receptor (SEQ ID NO: 4) | | | | | | | | |
| P02656 | Apolipoprotein C-III (SEQ ID NO: 5) | 2.3 | 7.5 | 3.3 | 0.0019 | <0.0001 | <0.0001 | <0.0001 | 1 < 2 < 3 |
| P01243 | Chorionic somatomammotropin | 5.2 | 7.4 | 1.4 | 0.0041 | 0.0002 | 0.3100 | 0.0049 | 1 < 2 < 3 |
| | hormone & Somatotropin (SEQ ID | | | | | | | | |
| | NO: 6) | | | | | | | | |
| P29400 | Collagen alpha-5(IV) chain (SEQ ID | −1.1 | 6.4 | 7.2 | | 0.0090 | 0.0079 | <0.0001 | 1~2 < 3 |
| | NO: 7) | | | | | | | | |
| P11464, | Pregnancy specific glyprotein | 2.2 | 6.2 | 2.9 | <0.0001 | <0.0001 | <0.0001 | <0.0001 | 1 < 2 < 3 |
| P11465, | 1, 2, 4, 7, 9 precursors (SEQ ID NO: 8), | | | | | | | | |
| Q00888, | (SEQ ID NO: 9), (SEQ ID NO: 10), | | | | | | | | |
| Q13046, | (SEQ ID NO: 11), (SEQ ID NO: 12) | | | | | | | | |
| Q00887 ^{d} | | | | | | | | | |
| P18428 | Lipopolysaccharide-binding protein | 4.4 | 5.5 | 1.2 | 0.0130 | 0.0025 | 0.5800 | 0.0134 | 1 < 2 < 3 |
| | (SEQ ID NO: 13) | | | | | | | | |
| Q9UGR4 | Fibulin-1 (SEQ ID NO: 14) | 2.3 | 5.2 | 2.2 | 0.0001 | <0.0001 | <0.0001 | <0.0001 | 1 < 2 < 3 |
| P02655 | Apolipoprotein C-II (SEQ ID NO: 15) | −1.1 | 4.2 | 4.8 | 0.8100 | <0.0001 | <0.0001 | <0.0001 | 1~2 < 3 |
| P13727 | Bone-marrow proteoglycan (SEQ ID | 4.4 | 4.2 | −1.0 | <0.0001 | <0.0001 | 0.8100 | <0.0001 | 1 < 2~3 |
| | NO: 16) | | | | | | | | |
| P02671 | Fibrinogen alpha chain (SEQ ID | −8.1 | −2.0 | 4.0 | 0.0002 | 0.0690 | 0.0390 | | |
| | NO: 17) | | | | | | | | |
| P20742 | Pregnancy zone protein (SEQ ID | 3.0 | 3.2 | 1.1 | <0.0001 | <0.0001 | 0.1500 | <0.0001 | 1 < 2~3 |
| | NO: 18) | | | | | | | | |
| P02652 | Apolipoprotein A-II (SEQ ID NO: 19) | 1.4 | 3.2 | 2.3 | 0.5600 | 0.0091 | 0.0330 | 0.0065 | 1 < 2 < 3 |
| Q13822 | Ectonucleotide | −1.0 | 3.1 | 3.1 | 0.9800 | 0.0018 | 0.0012 | 0.0010 | 1~2 < 3 |
| | pyrophosphatase/phosphodiesterase | | | | | | | | |
| | (SEQ ID NO: 20) | | | | | | | | |
| P02787 | Serotransferrin (SEQ ID NO: 21) | 1.7 | 2.7 | 1.6 | <0.0001 | <0.0001 | <0.0001 | <0.0001 | 1 < 2 < 3 |
| O14791 | Apolipoprotein-L1 (SEQ ID NO: 22) | −1.6 | 1.6 | 2.7 | 0.2500 | 0.1200 | 0.0060 | 0.0499 | 1 > 2 < 3 |
| Q7Z7G0 | Target of Nesh-SH3 (SEQ ID NO: 23) | −5.6 | −2.2 | 2.5 | 0.0230 | 0.2600 | | | |
| Q08380 | Galectin-3-binding protein (SEQ ID | 1.2 | 2.3 | 1.9 | 0.4700 | 0.0004 | 0.0029 | 0.0001 | 1~2 < 3 |
| | NO: 24) | | | | | | | | |
| P02649 | Apolipoprotein E (SEQ ID NO: 25) | −1.2 | 1.9 | 2.2 | 0.4400 | 0.0002 | <0.0001 | <0.0001 | 1~2 < 3 |
| P04275 | von Willebrand factor (SEQ ID NO: 26) | −1.0 | 2.2 | 2.2 | 0.9900 | <0.0001 | <0.0001 | <0.0001 | 1~2 < 3 |
| P08185 | Corticosteroid-binding globulin (SEQ | 2.1 | 2.0 | −1.0 | <0.0001 | <0.0001 | 0.7800 | <0.0001 | 1 < 2~3 |
| | ID NO: 27) | | | | | | | | |
| Q96IY4 | Carboxypeptidase B2 (SEQ ID NO: 28) | 1.9 | 1.8 | −1.1 | 0.0075 | 0.0180 | 0.7300 | 0.0039 | 1 < 2~3 |
| P01233 | Choriogonadotropin subunit beta | −5.1 | −2.7 | 1.9 | <0.0001 | 0.0022 | 0.1500 | 0.0121 | 1 > 2 < 3 |
| | (SEQ ID NO: 29) | | | | | | | | |
| O75636 | Ficolin-3 (SEQ ID NO: 30) | 1.3 | 1.8 | 1.4 | 0.2200 | 0.0055 | 0.0980 | 0.0008 | 1 < 2 < 3 |
| Q76LX8 | ADAMTS-13 (SEQ ID NO: 31) | −4.1 | −2.2 | 1.8 | 0.0300 | 0.2000 | | | |
| P02647 | Apolipoprotein A-I (SEQ ID NO: 32) | 1.5 | 1.8 | 1.2 | 0.0008 | <0.0001 | 0.1100 | <0.0001 | 1 < 2 < 3 |
| P26927 | Hepatocyte growth factor-like protein | −2.4 | −1.4 | 1.8 | 0.0071 | 0.2800 | 0.0920 | | |
| | (SEQ ID NO: 33) | | | | | | | | |
| Q8IVI8 | Fibronectin (SEQ ID NO: 34) | −1.2 | 1.4 | 1.8 | 0.0410 | <0.0001 | <0.0001 | <0.0001 | 1~2 < 3 |
| P80108 | Phosphatidylinositol-glycan-specific | 1.3 | 1.7 | 1.4 | 0.2300 | 0.0034 | 0.0670 | 0.0003 | 1 < 2 < 3 |
| | phospholipase D (SEQ ID NO: 35) | | | | | | | | |
| P01009 | Alpha-1-antitrypsin (SEQ ID NO: 36) | 1.7 | 1.5 | −1.1 | <0.0001 | <0.0001 | 0.0180 | <0.0001 | 1 < 2 > 3 |
| P15169 | Carboxypeptidase N catalytic chain | 1.7 | −1.1 | −1.9 | 0.0069 | 0.6900 | 0.0012 | | |
| | (SEQ ID NO: 37) | | | | | | | | |
| Q04756 | Hepatocyte growth factor activator | −3.8 | −2.3 | 1.7 | 0.0098 | 0.0530 | 0.5100 | | |
| | (SEQ ID NO: 38) | | | | | | | | |
| P04180 | Phosphatidylcholine-sterol | 1.7 | −1.1 | −1.8 | 0.0850 | 0.8200 | 0.0420 | | |
| | acyltransferase (SEQ ID NO: 39) | | | | | | | | |
| Q13787 | Apolipoprotein B-100 (SEQ ID NO: 40) | 1.3 | 1.7 | 1.3 | <0.0001 | <0.0001 | <0.0001 | <0.0001 | 1 < 2 < 3 |
| P03952 | Plasma kallikrein (SEQ ID NO: 41) | −1.4 | 1.1 | 1.6 | 0.0058 | 0.3900 | 0.0002 | 0.0350 | 1 > 2 < 3 |
| P43652 | Afamin (SEQ ID NO: 42) | 1.3 | 1.6 | 1.2 | 0.0068 | <0.0001 | 0.0460 | <0.0001 | 1 < 2 < 3 |
| P06727 | Apolipoprotein A-IV (SEQ ID NO: 43) | −1.5 | 1.1 | 1.6 | 0.0002 | 0.4800 | <0.0001 | 0.0216 | 1 > 2 < 3 |
| P17936 | Insulin-like growth factor-binding | −1.8 | −1.2 | 1.5 | 0.0170 | 0.4500 | 0.0900 | | |
| | protein 3 (SEQ ID NO: 44) | | | | | | | | |
| P08571 | Monocyte differentiation antigen | −2.4 | −1.6 | 1.5 | 0.0033 | 0.0760 | 0.2100 | | |
| | CD14 (SEQ ID NO: 45) | | | | | | | | |
| P06276 | Cholinesterase (SEQ ID NO: 46) | −2.2 | −1.6 | 1.4 | 0.0006 | 0.0360 | 0.1500 | | |
| P08697 | Alpha-2-antiplasmin (SEQ ID NO: 47) | −1.6 | −1.2 | 1.4 | 0.0500 | 0.4500 | 0.2100 | | |
| P02747 | Complement C1q subcomponent | −1.6 | −1.2 | 1.3 | 0.0270 | 0.4400 | 0.1400 | | |
| | subunit C (SEQ ID NO: 48) | | | | | | | | |
| P05452 | Tetranectin (SEQ ID NO: 49) | −2.6 | −2.0 | 1.3 | <0.0001 | 0.0004 | 0.2600 | 0.0062 | 1 > 2 < 3 |
| Q02388 | Collagen alpha-1(VII) chain (SEQ ID | 1.3 | −3.9 | −5.1 | 0.7300 | | 0.0290 | <0.0001 | 1 < 2 > 3 |
| | NO: 50) | | | | | | | | |
| P00738 | Haptoglobin & Haptoglobin repated | −1.9 | −1.5 | 1.3 | <0.0001 | <0.0001 | 0.0130 | 0.0048 | 1 > 2 < 3 |
| | protein (SEQ ID NO: 51) | | | | | | | | |
| P09172 | Dopamine beta-hydroxylase (SEQ ID | −2.4 | −1.9 | 1.2 | 0.0400 | 0.1000 | 0.6300 | | |
| | NO: 52) | | | | | | | | |
| P07358 | Complement component C8 beta | −1.6 | −1.3 | 1.2 | 0.0001 | 0.0077 | 0.1600 | | |
| | chain (SEQ ID NO: 53) | | | | | | | | |
| P02745 | Complement C1q subcomponent | 1.1 | −1.9 | −2.1 | 0.6700 | 0.0830 | 0.0280 | | |
| | Subunit A (SEQ ID NO: 54) | | | | | | | | |
| P49908 | Selenoprotein P (SEQ ID NO: 55) | −2.2 | −2.0 | 1.1 | 0.0470 | 0.0690 | 0.8400 | | |
| P43121 | Cell surface glycoprotein MUC18 | −4.1 | −4.2 | −1.0 | 0.0300 | 0.0290 | | | |
| | (SEQ ID NO: 56) | | | | | | | | |
| P41222 | Prostaglandin-H2 D-isomerase (SEQ | −4.1 | −4.2 | −1.0 | 0.0300 | 0.0290 | | | |
| | ID NO: 57) | | | | | | | | |
| P00915 | Carbonic anhydrase 1 (SEQ ID | −2.3 | −2.5 | −1.1 | 0.0014 | 0.0005 | 0.8000 | 0.0049 | 1 > 2~3 |
| | NO: 58) | | | | | | | | |
| P06396 | Gelsolin (SEQ ID NO: 59) | −1.5 | −1.7 | −1.1 | <0.0001 | <0.0001 | 0.1100 | <0.0001 | 1 > 2 > 3 |
| P07996 | Thrombospondin-1 (SEQ ID NO: 60) | −1.4 | −1.6 | −1.2 | 0.0067 | 0.0002 | 0.3000 | 0.0099 | 1 > 2 > 3 |
| Q6P3U9 | 14-3-3 protein zeta/delta (SEQ ID | −2.2 | −2.7 | −1.2 | 0.0820 | 0.0400 | 0.7500 | | |
| | NO: 61) | | | | | | | | |
| P04196 | Histidine-rich glycoprotein (SEQ ID | −1.6 | −2.2 | −1.4 | <0.0001 | <0.0001 | 0.0046 | <0.0001 | 1 > 2 > 3 |
| | NO: 62) | | | | | | | | |
| P32119 | Peroxiredoxin-2 (SEQ ID NO: 63) | −3.3 | −5.0 | −1.5 | 0.0045 | 0.0005 | 0.5000 | 0.0056 | 1 > 2 > 3 |
| P07737 | Profilin-1 (SEQ ID NO: 64) | −1.9 | −3.3 | −1.8 | 0.1400 | 0.0280 | 0.3400 | 0.0461 | 1 > 2 > 3 |
| P14151 | L-selectin (SEQ ID NO: 65) | −2.3 | −4.2 | −1.8 | 0.0230 | 0.0009 | 0.2600 | 0.0065 | 1 > 2 > 3 |
| Q15485 | Ficolin-2 (SEQ ID NO: 66) | −3.1 | −5.8 | −1.9 | 0.1100 | 0.0210 | | <0.0001 | 1 > 2 > 3 |
| O95479 | GDH/6PGL endoplasmic bifunctional | −3.1 | −5.8 | −1.9 | 0.1100 | 0.0210 | | <0.0001 | 1 > 2 > 3 |
| | protein (SEQ ID NO: 67) | | | | | | | | |
| P02741 | C-reactive protein (SEQ ID NO: 68) | −2.7 | −5.3 | −1.9 | 0.0790 | 0.0077 | | 0.0371 | 1 > 2 > 3 |

^{a} The fold expression change of protein was quantitated using the formula described in ref. {Old, 2005 #29}. Proteins with significant (p-value <= 0.05 as highlighted in bold font and a fold change of >=±1.5) differential expression in any pair-wise comparisons are listed in table 2 above with their Swiss-Prot accessions.
^{b} Trends in the protein expression changes with respective to gestational age is tested using a Orthogonal polynomial trend test.
^{c} Notation: 1 - first trimester, 2 - second trimester, 3 - 3rd trimester, > - up regulation, < - down regulation, ~ - no significant change.
^{d} Proteins that shared significant sequence homology are collapsed and treated as a single entry.

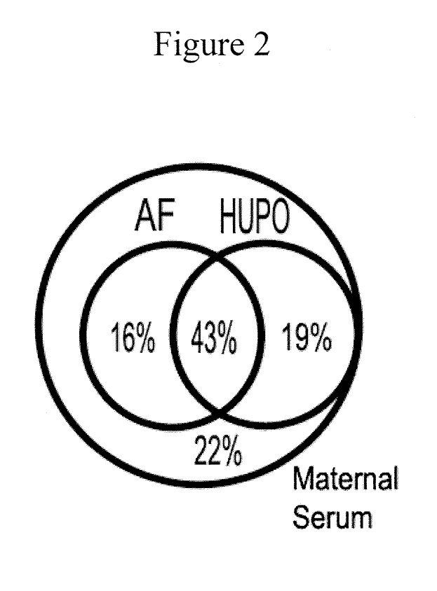

Proteins are functionally annotated using gene ontology (GO) annotations from NCBI database. Annotations are further inspected to mark the proteins involved in complement cascade, coagulation cascade, and pregnancy accordingly. The total functional composition of maternal serum proteome is shown in FIG. 2. Metabolic (21%), catalytic (13%), and defense response (13%) proteins constitute majority of molecules found in maternal serum. Complement cascade (9%), coagulation cascade (7%) and pregnancy associated (4%) proteins also contributed to the over all composition of maternal serum. A fairly good number of proteins (12%) did not have any appropriate functional annotations.

Human serum and amniotic fluid (AF) are highly studied proteomes due their clinical significance. We cross-referenced the maternal plasma proteins found in this study to existing plasma and AF proteomes. High-confident and nonredundant known plasma proteome was derived by combining serum proteins reported in HUPO plasma proteome {States, 2006 #68} and Anderson et. al {Anderson, 2004 #67} using procedure outlined in reference {Dasari, 2007 #38}. It should be noted that the comprehensive known plasma proteome contains both maternal and non-maternal proteins. A comprehensive and nonredundant amniotic fluid (AF) proteome was also generated by combining the AF proteins reported in Cho C. K et. al {Cho, 2007 #64} and Michaels J-E. A. et. al. {Michaels, 2007 #63}. Maternal serum proteins found in this study were cross-referenced with the high-confident known plasma and AF proteomes. The percent overlap between the three proteomes is shown in FIG. 2. Among a total of 266 maternal serum proteins found in this study, 116 (43%) are also found in both known plasma and AF proteomes, 51 (19%) were found in known plasma proteome, 43 (16%) were found in known AF proteome, and rest of 56 (22%) are uniquely detectable in maternal plasma. A majority of the maternal plasma proteins were confirmed by known plasma and AF proteomes.

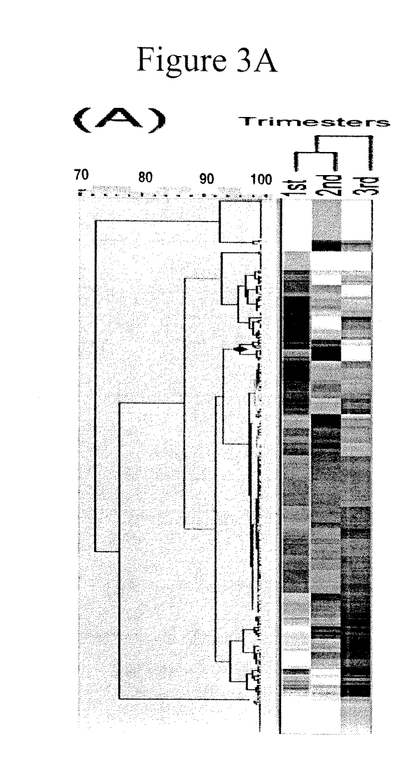

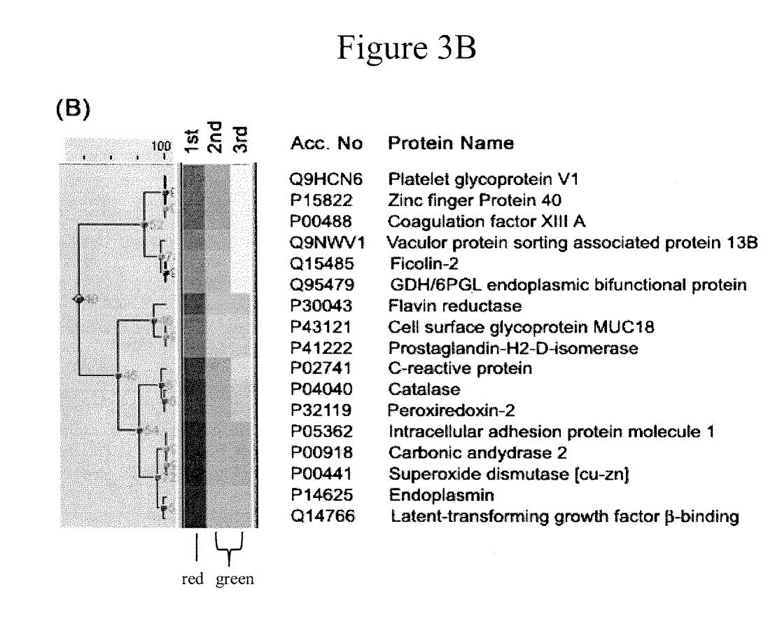

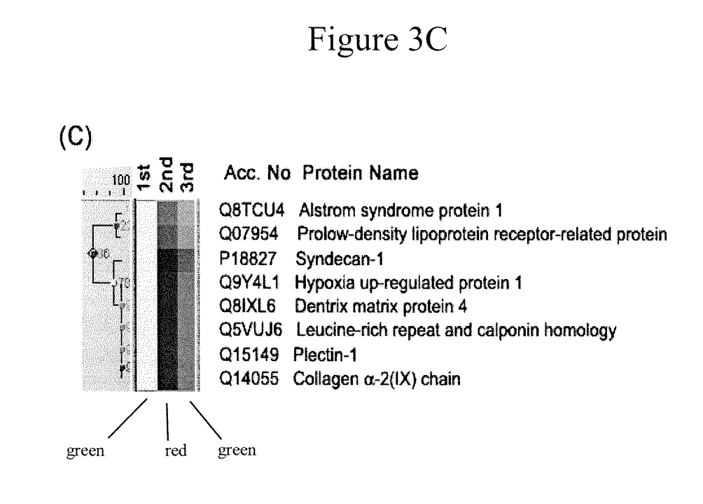

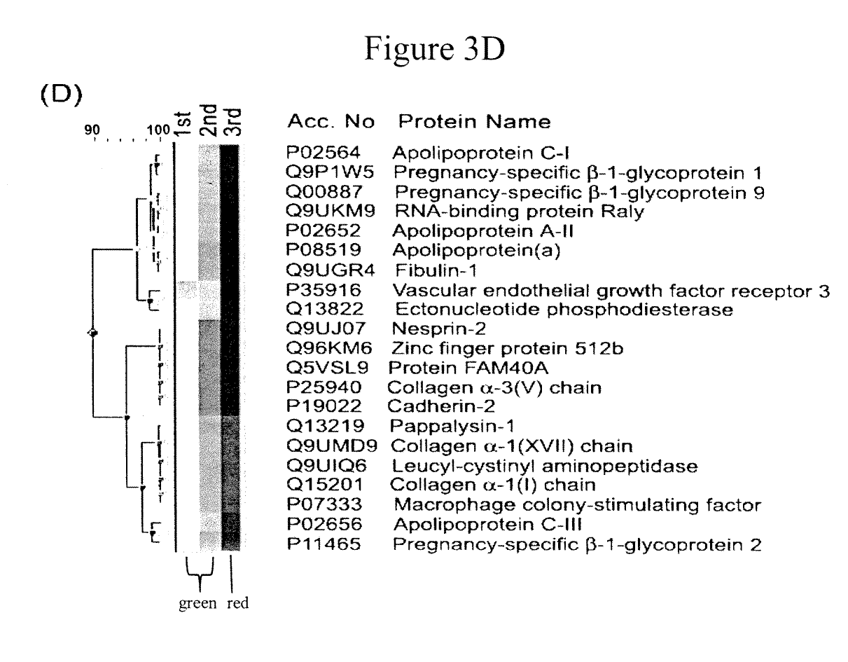

Total number of MS/MS spectra matched to a protein is directly related to its abundance in complex mixtures {Liu, 2004 #39}. Global protein expression changes in maternal serum during pregnancy are visualized using GeneMaths software (version 1.5, Applied Maths, Austin, Tex.). Spectral counts of proteins with at least two peptide identifications (p>0.8) in at least one of the trimester samples were individually mean normalized and loaded into GeneMaths software. Proteins with similar expression changes between trimester samples were both hierarchically and vertically clustered using Euclidean distance learning method with 200 simulations (see FIG. 3a). Hierarchal cluster analysis showed that a majority of differentially expressed proteins are highly up regulated either during 1^{st}, 2^{nd}, or 3^{rd} trimesters. Representative protein clusters that show afore-mentioned expression trends are illustrated in FIG. 3b, FIG. 3c, and FIG. 3d, respectively. Vertical cluster analysis showed that overall protein expression profiles of 1^{st} and 2^{nd} trimester maternal serum are similar to each other when compared to those of 3^{rd} trimester.

Spectral counts of proteins were also subjected to a highly sensitive label-free quantitation (a.k.a spectral counting) method to rapidly determine differentially expressed proteins between complex mixtures. Maternal plasma proteins with at least three unique and confident (probability ≧0.8) peptide identifications in one of the samples were subjected to label-free quantitation (see methods). In total, three independent pair-wise comparisons were performed for each protein: first vs. second trimester, first vs. third trimester and second vs. third trimester. Proteins with a relative expression change of ≧1.5 fold and a p-value ≦0.05 in any of the comparisons were considered as potentially differentially expressed between the samples. The number of decoy sequences that passed the above-mentioned criteria is used to estimate the false positive rate of the label-free quantitation technique. A total of 64 serum proteins (shown in Table 2) and two decoy proteins passed the label-free quantitation method. Hence, the false positive rate of the label-free technique used in this study is estimated as 3%. Proteins are also subjected to a label-free trend test to catch significant trends in their expression change during the progression of pregnancy (see methods). Proteins that passed the trend test are annotated with the trend and its p-value as shown in last two columns of the Table 2. A total of 18 proteins (28%) showed an increasing trend to term, 9 proteins (14%) showed a decreasing trend to term, 7 (11%) showed a decreasing trend from first to second trimester and increasing trend there on to term, and 2 (3%) showed an increasing trend between first and second trimester and decreasing trend from there on to term.

Label-free quantitation is a very thorough and also time-consuming process. It is often customary to utilize the method to discover large differences between modest numbers of samples. Hence, all the proteins that passed the technique need to be validated on a large set of samples using other absolute protein concentration measurement techniques. In this study, as a proof of concept, a total of 5 proteins (CSH, PSG1, βHCG, PAPP-A, and ApoC-III) that passed label-free quantitation were validated with ELISA on a cohort of 44 healthy maternal subjects. Measured protein concentrations were log-transformed and compared in a pair-wise fashion between first vs. second, first vs. third and, second vs. third trimesters, respectively, using an ANOVA test. Proteins that passed at least one pair-wise comparison are shown below in Table 3. The mean concentration of each protein in respective trimesters was determined by computing the harmonic mean concentration (ng/ml) measured by ELISA (shown in Table 3). Proteins that passed label-free quantitation also passed the ELISA quantitation method. This observation underscores the utility of using label-free quantitation to determine potential biomarkers in complex mixtures of small sample sizes and rapidly cross validating them on a larger sample set using an absolute quantitation technique like ELISA or mass spectrometry based method (Metabolic labeling, isotopic labeling, and MRM) with internal standards.

TABLE 3
Validation of Label-Free Pregnancy Associated Biomarkers with ELISA

| | | Harmonic Mean | | | | | | | | |
|---|---|---|---|---|---|---|---|---|---|---|
| | | Concentration in Trimesters | | | 1st vs. 2nd | | 1st vs. 3rd | | 2nd vs. 3rd | |
| Swiss-Prot | | (ng/ml) ^{b} | | | Trimester | | Trimester | | Trimester | |
| Accession ^{a} | Description | 1st | 2nd | 3rd | p-value ^{c} | AUROC | p-value | AUROC | p-value | AUROC |
| P01243 | Chorionic | 1891.7 | 56153.7 | 123123.7 | <0.0001 | 1 | <0.0001 | 0.976 | <0.0001 | 0.92 |
| | somatomammotropin | | | | | | | | | |
| | hormone (SEQ ID NO: 6) | | | | | | | | | |
| P11464 | Pregnancy-specific beta-1- | 5393.8 | 16026.8 | 33053.2 | 0.0001 | 0.978 | 0.0365 | 0.998 | <0.0001 | 0.905 |
| | glycoprotein 1 (SEQ ID | | | | | | | | | |
| | NO: 8) | | | | | | | | | |
| P01233 | Choriogonadotropin | 406.0 | 19.9 | 24.1 | <0.0001 | 0.982 | <0.0001 | 0.968 | 0.4846 | 0.528 |
| | subunit beta (SEQ ID | | | | | | | | | |
| | NO: 29) | | | | | | | | | |
| Q13219 | Pappalysin-1 (SEQ ID | 4586.6 | 141153.7 | 397303.6 | 0.0003 | 0.993 | 0.0026 | 0.997 | <0.0001 | 0.804 |
| | NO: 1) | | | | | | | | | |
| P02656 | Apolipoprotein C-III (SEQ | 161309.6 | 143444.4 | 203304.0 | 0.1734 | 0.589 | 0.0599 | 0.712 | 0.0052 | 0.784 |
| | ID NO: 5) | | | | | | | | | |

Selected proteins markers from label-free quantitation are validated using ELISA experiments. Proteins are listed according to their Swiss-Prot accessions
^{a} harmonic mean of concentrations measured in respective trimesters
^{b} p-value from corresponding ANOVA test
^{c} and its area under the ROC (AUROC).

The utility of a protein biomarker depends on its power to robustly distinguish between patient populations. The absolute concentrations of proteins from the ELISA experiment were subjected to logistic regression followed by an AUROC analysis (shown in Table 3).

Discussion

Serum proteome from first, second and third trimester healthy human maternal subjects was sequenced using tandem mass spectrometry. Functional annotation of the proteome uncovered a large number of metabolic, defense response, complement cascade, coagulation cascade, and pregnancy associated proteins present in maternal serum. This suggests that a majority of maternal serum proteins are involved in maternal and fetal development, innate immune defense, and hemostasis, which are important physiological functions of serum during gestation. A majority of the maternal serum proteins (59%) were also found in amniotic fluid (AF) proteome. This supports the hypothesis that serial assessment of easily accessible body fluids like serum could be used instead of high risk amniocentesis for maternal-fetal diagnostics. Maternal serum protein expression profiles from all trimesters were subjected to both hierarchical and vertical clustering. Hierarchical clustering showed that most of the differentially expressed maternal serum proteins are highly up regulated during only one of the trimesters. Vertical clustering showed that protein expression profiles of 1^{st} and 2^{nd} trimester serum are closely related than of 3^{rd} trimester.

Maternal serum proteins were also subjected to a more sensitive label-free quantification method and a total of 67 proteins were identified as significantly differentially expressed between any two trimesters. This highlights the utility of using large-scale protein identification and quantitation technologies (proteomics) for rapidly identifying proteome wide differences between complex biological samples. Results of label-free quantitation are successfully validated with traditional ELISA technique. Hence, it is possible to envision a holistic mass spectrometry based assay platform for biomarker based disease diagnostics.

We observed four specific types of protein expression changes in maternal serum between first to third trimester: continuous up regulation to term, continuous down regulation to term, up regulation from 1^{st} to 2^{nd} trimester and slowing down there after to term, down regulation from 1^{st} to 2^{nd} trimester and slowing down there after to term, and up regulation from 1^{st} to 2^{nd} trimester and down regulation back to term.

Several pregnancy-specific and placental specific proteins showed a continuous up regulation of their expression during gestation. Pappalysin-A (PAPP-A) is a placental-specific glycoprotein that plays an important role during pregnancy by slowing down the transformation of maternal lymphocytes into lymphoblasts {Grudzinskas, 1982 #79}. A continuous up regulation of PAPP-A with gestation prevents immuno-rejection of maturing fetus by maternal adaptive immune system. Interestingly, we have not observed a high expression of PAPP-A during first trimester. Such an early expression of PAPP-A has been correlated to development of fetal aneuploidy {Malone, 2005 #84; Dugoff, 2005 #87}. Chorionic somatomammotropin hormone (CSH) and various forms of pregnancy-specific-β-1-glycoproteins (PSGs) also showed a similar trend in their expression. CSH has been known to function as an immuno-suppression, lactogenetic, and erythropoietic agent {Grudzinskas, 1982 #79}. PSGs contribute to immuno-suppression of maternal adaptive immune system towards maturing fetus {Grudzinskas, 1982 #79}. Hence, a continuous up regulation of placental and pregnancy specific proteins is vital to the development and protection of the fetus.

Histidine-rich glycoprotein (HRG) and C-reactive protein (CRP) are a few proteins that showed a continuous down regulation towards term. HRG is a fibrinolysis inhibitor {Leebeek, 1989 #105} whose down regulation enhances blood coagulation and fibrinolysis {Tsuchida-Straeten, 2005 #106} making maternal blood hypercoagulable. CRP is an acute phase immune response molecule and its down regulation is necessary to prevent immuno-rejection of maturing fetus. Elevated levels of CRP have been linked to spontaneous preterm birth with subclinical infection {Gibbs, 1992 #112}. These observations suggest that anti-fibrinolytic and pro-inflammatory agents in maternal serum have to be continuously down regulated to term for a favorable parturition outcome.

Pregnancy zone protein (PZP), Corticosteroid-binding globulin (CBG), and Bone-marrow proteoglycan 2 (Eosinophil granule major basic protein, MBP) showed up regulation from first to second trimester and slowed down there after to term. PZP and CBG are one of the major oestrogen inducing proteins whose expression is known to follow the observed trend {Grudzinskas, 1982 #79}. The perfect agreement between experimental data and known literature shows the credibility of this study. MBP is a major physiological inhibitor of PAPP-A {Overgaard, 2000 #113}. It is interesting to note that observed trend of MBP expression perfectly correlates with observed trend of PAPP-A expression in this study. Hence, we speculate that somewhere between first and second trimesters, MBP expression slows down setting the stage for up regulation of PAPP-A. The mechanism(s) by which this switch happens is yet unknown.

Human choriogonadotropin subunit β (βHCG), a placental-specific protein with a strong luteotropic function, showed down regulation from first to second trimester and slowing down there after to term. βHCG plays a vital role in maintaining the function of corpus leteum during early stages of pregnancy {Grudzinskas, 1982 #79}. Recently, βHCG has also been observed to play a role as an endogenous tocolytic agent in normal pregnancy. In agreement with the above facts, βHCG expression, observed in this study, was high during first trimester signaling viable trophoblasts {Licht, 2001 #110; Grudzinskas, 1982 #79} and decreased with progression of gestation contributing to increase in contractility of uterine muscle in order to gradually prepare for onset of labor {Edelstam, 2007 #109}. Abnormal levels of βHCG have been associated with Down syndrome {Malone, 2005 #84; Dugoff, 2005 #87}, preterm birth, preeclampsia, and still birth {Towner, 2006 #111}.

This is the first study to uncover significant proteome wide differences in maternal serum between first, second and third trimester human subjects. This dataset establishes the expression patterns of maternal proteins during healthy gestation. The observed expression patterns play a vital role in placental implantation, fetal maturity, fetal protection, lactogensis, hypercoagulation of maternal plasma, and parturition. Any deviations from the observed expression patterns were associated with placental pathology, fetal aneuploidy, preterm birth, and preeclampsia. The role of many other proteins, found in this study, and their expression trends during gestation is still unknown. The protein expression patterns identified in this study lay a critical foundation for development of biomarker based diagnostics in maternal-fetal medicine.

### Example 2

### Maternal serum proteome profile of early placentation in IVF is Distinct from Normal Placentation

Placentation following in vitro fertilization (IVF) may differ from normal placentation and result in differences in placental proteins detected in maternal serum in prenatal screening. In order to identify differences in pregnancy protein expression between IVF and normal pregnancies, the maternal serum proteome in early pregnancy was characterized.

Study Design: A total of 110 women (55 following IVF and 55 with spontaneous pregnancy) from a prospective observational cohort were included. Maternal serum samples were collected at 11 and 19 gestational weeks. Proteome analysis was performed using fluorescence 2-D gel electrophoresis (2-DIGE), multidimensional liquid chromatography tandem mass spectrometry (2D LC-MS/MS) and label-free quantification (spectral counting). Pair-wise comparison was performed using χ² goodness-of-fit tests. Statistical significance for each protein was determined after adjusting for multiple comparisons via the false-discovery rate (FDR) method. Immunoassays were used for accurate quantification and evaluated using the Receiver Operating Characteristic (ROC) curves.

Results: Gestational age at delivery or perinatal outcome parameters did not differ between the groups. 2D-DIGE analysis identified a distinct differential expression pattern between IVF and normal groups. 2D LC-MS/MS analysis identified 368 unique proteins. Protein expression differences were noted in extra-cellular matrix proteins, cytoskeletal, vascular, complement and transport proteins; all are important in placentation. Pregnancy specific glycoprotein-1 (PSG1) (SEQ ID NO:8), somatomammotrophin-1 (SEQ ID NO:6), and lipopolysaccharide binding protein (SEQ ID NO:13) showed the most significant differences at 11 weeks of gestation. Most proteins reached equal expression in both groups by 19 weeks. Only PSG1 remained significantly different at 19 weeks. Commonly measured pregnancy proteins (pregnancy associated plasma protein-1 (SEQ ID NO:1), chorionic gonadotropin (SEQ ID NO:29), endoglin (SEQ ID NO:69), fibronectin (SEQ ID NO:34)) had similar trends from 11 to 19 weeks in both groups.

Conclusions: First trimester maternal serum proteome analyses identified distinct differences in protein detection between IVF and spontaneous pregnancies that persisted until mid-gestation. These findings may help explain adverse pregnancy outcomes associated with IVF pregnancy and suggest early, selective treatments.

Throughout the foregoing description the invention has been discussed with reference to certain embodiments, but it is not so limited. Indeed, various modifications of the invention in addition to those shown and described herein will become apparent to those skilled in the art from the foregoing description and fall within the scope of the appended claims.

All references cited throughout the description, and the references cited therein, are hereby expressly incorporated by reference in their entirety.

### REFERENCES

Alan, A. (1990). Categorical Data Analysis, John Wiley and Sons: 100-102.
Anderson, N. L., M. Polanski, et al. (2004). “The human plasma proteome: a nonredundant list developed by combination of four separate sources.” Mol Cell Proteomics 3(4): 311-26.
Battaglia, F. C. (1992). “New concepts in fetal and placental amino acid metabolism.” J Anim Sci 70(10): 3258-63.
Bischof, P. (1981). “Pregnancy-associated plasma protein-A: an inhibitor of the complement system.” Placenta 2(1): 29-34.
Bohles, H. (1997). “Antioxidative vitamins in prematurely and maturely born infants.” Int J Vitam Nutr Res 67(5): 321-8.
Cho, C. K., S. J. Shan, et al. (2007). “Proteomics analysis of human amniotic fluid.” Mol Cell Proteomics 6(8): 1406-15.
Clark, M. F. and A. N. Adams (1977). “Characteristics of the microplate method of enzyme-linked immunosorbent assay for the detection of plant viruses.” J Gen Virol 34(3): 475-83.
Coleman, R. A. (1986). “Placental metabolism and transport of lipid.” Fed Proc 45(10): 2519-23.
Cross, J. C. (2003). “The genetics of pre-eclampsia: a feto-placental or maternal problem?” Clin Genet. 64(2): 96-103.
Dasari, S., L. Pereira, et al. (2007). “Comprehensive proteomic analysis of human cervical-vaginal fluid.” J Proteome Res 6(4): 1258-68.
Dolan, S. M., S. J. Gross, et al. (2007). “The contribution of birth defects to preterm birth and low birth weight.” Obstet Gynecol 110(2 Pt 1): 318-24.
Dugoff, L., J. C. Hobbins, et al. (2005). “Quad screen as a predictor of adverse pregnancy outcome.” Obstet Gynecol 106(2): 260-7.
Edelstam, G., C. Karlsson, et al. (2007). “Human chorionic gonadatropin (hCG) during third trimester pregnancy.” Scand J Clin Lab Invest 67(5): 519-25.
Fenyo, D. and R. C. Beavis (2003). “A method for assessing the statistical significance of mass spectrometry-based protein identifications using general scoring schemes.” Anal Chem 75(4): 768-74.
Gibbs, R. S., R. Romero, et al. (1992). “A review of premature birth and subclinical infection.” Am J Obstet Gynecol 166(5): 1515-28.
Grudzinskas, J. G., B. Teisner, et al. (1982). Pregnancy Proteins: Biology, Chemistry and Clinical Applications, Academic Press.
Holmes, V. A. and J. M. Wallace (2005). “Haemostasis in normal pregnancy: a balancing act?” Biochem Soc Trans 33(Pt 2): 428-32.
Hosmer, D. W. and S. Lemeshow (2000). Applied Logistic Regression, John Wiley and Sons.
Hubert, L. J. (1973). “The Use of Orthogonal Polynomials for Trend Analysis.” American Educational Research Journal 10(3): 241-244.
Knobloch, V. and I. Miler (1988). “The development of immunological relationship between mother and fetus under physiological and pathological conditions.” Allerg Immunol (Leipz) 34(4): 219-31.
Leebeek, F. W., C. Kluft, et al. (1989). “Histidine-rich glycoprotein is elevated in mild liver cirrhosis and decreased in moderate and severe liver cirrhosis.” J Lab Clin Med 113(4): 493-7.
Licht, P., V. Russu, et al. (2001). “On the role of human chorionic gonadotropin (hCG) in the embryo-endometrial microenvironment: implications for differentiation and implantation.” Semin Reprod Med 19(1): 37-47.
Link, A. J., J. Eng, et al. (1999). “Direct analysis of protein complexes using mass spectrometry.” Nat Biotechnol 17(7): 676-82.
Liu, H., R. G. Sadygov, et al. (2004). “A model for random sampling and estimation of relative protein abundance in shotgun proteomics.” Anal Chem 76(14): 4193-201.
Lockwood, C. J., P. Toti, et al. (2007). “Thrombin regulates soluble fms-like tyrosine kinase-1 (sFlt-1) expression in first trimester decidua: implications for preeclampsia.” Am J Pathol 170(4): 1398-405.
Malone, F. D., J. A. Canick, et al. (2005). “First-trimester or second-trimester screening, or both, for Down's syndrome.” N Engl J Med 353(19): 2001-11.
Michaels, J. E., S. Dasari, et al. (2007). “Comprehensive proteomic analysis of the human amniotic fluid proteome: gestational age-dependent changes.” J Proteome Res 6(4): 1277-85.
Michel, P. E., D. Crettaz, et al. (2006). “Proteome analysis of human plasma and amniotic fluid by Off-Gel isoelectric focusing followed by nano-LC-MS/MS.” Electrophoresis 27(5-6): 1169-81.
Muller, P. R., A. H. James, et al. (2006). “Circulating angiogenic factors and abnormal uterine artery Doppler velocimetry in the second trimester.” Hypertens Pregnancy 25(3): 183-92.
Nerurkar, L. S., M. Namba, et al. (1984). “Rapid detection of herpes simplex virus in clinical specimens by use of a capture biotin-streptavidin enzyme-linked immunosorbent assay.” J Clin Microbiol 20(1): 109-14.
Nesvizhskii, A. I., A. Keller, et al. (2003). “A statistical model for identifying proteins by tandem mass spectrometry.” Anal Chem 75(17): 4646-58.
Old, W. M., K. Meyer-Arendt, et al. (2005). “Comparison of label-free methods for quantifying human proteins by shotgun proteomics.” Mol Cell Proteomics 4(10): 1487-502.
Overgaard, M. T., J. Haaning, et al. (2000). “Expression of recombinant human pregnancy-associated plasma protein-A and identification of the proform of eosinophil major basic protein as its physiological inhibitor.” J Biol Chem 275(40): 31128-33.
Pang, J. X., N. Ginanni, et al. (2002). “Biomarker discovery in urine by proteomics.” J Proteome Res 1(2): 161-9.
Pepe, M. S. (2003). The statistical evaluation of medical tests for classification and prediction, Oxford University Press.
Pereira, L., A. P. Reddy, et al. (2007). “Identification of novel protein biomarkers of preterm birth in human cervical-vaginal fluid.” J Proteome Res 6(4): 1269-76.
Pieper, R., C. L. Gatlin, et al. (2003). “The human serum proteome: display of nearly 3700 chromatographically separated protein spots on two-dimensional electrophoresis gels and identification of 325 distinct proteins.” Proteomics 3(7): 1345-64.
Pitkin, R. M. (1985). “Calcium metabolism in pregnancy and the perinatal period: a review.” Am J Obstet Gynecol 151(1): 99-109.
Regnault, T. R., B. de Vrijer, et al. (2002). “Transport and metabolism of amino acids in placenta.” Endocrine 19(1): 23-41.
Salle, B. L., E. E. Delvin, et al. (2000). “Perinatal metabolism of vitamin D.” Am J Clin Nutr 71(5 Suppl): 1317S-24S.
Schander, K., W. Wahl, et al. (1979). “[The coagulation and fibrinolysis activities of the blood during various phases of labor].” Fortschr Med 97(38): 1696-702.
Sharkey, A. (1998). “Cytokines and implantation.” Rev Reprod 3(1): 52-61.
Slater, M. and C. R. Murphy (1999). “Differential expression of insulin-like growth factors in the uterine epithelium and extracellular matrix during early pregnancy.” Matrix Biol 18(6): 579-84.
Slowinski, T., H. H. Neumayer, et al. (2002). “Endothelin system in normal and hypertensive pregnancy.” Clin Sci (Lond) 103 Suppl 48: 446S-449S.
Spatling, L., G. Disch, et al. (1989). “Magnesium in pregnant women and the newborn.” Magnes Res 2(4): 271-80.
States, D. J., G. S. Omenn, et al. (2006). “Challenges in deriving high-confidence protein identifications from data gathered by a HUPO plasma proteome collaborative study.” Nat Biotechnol 24(3): 333-8.
Steele, C. D., R. J. Wapner, et al. (1996). “Prenatal diagnosis using fetal cells isolated from maternal peripheral blood: a review.” Clin Obstet Gynecol 39(4): 801-13.
Towner, D., S. Gandhi, et al. (2006). “Obstetric outcomes in women with elevated maternal serum human chorionic gonadotropin.” Am J Obstet Gynecol 194(6): 1676-81; discussion 1681-2.
Tsuchida-Straeten, N., S. Ensslen, et al. (2005). “Enhanced blood coagulation and fibrinolysis in mice lacking histidine-rich glycoprotein (HRG).” J Thromb Haemost 3(5): 865-72.
Van Lith, J. M., J. R. Beekhuis, et al. (1991). “Alpha-fetoprotein in fetal serum, amniotic fluid, and maternal serum.” Prenat Diagn 11(8): 625-8.
Washburn, M. P., D. Wolters, et al. (2001). “Large-scale analysis of the yeast proteome by multidimensional protein identification technology.” Nat Biotechnol 19(3): 242-7.
Xia, Y., C. C. Zhou, et al. (2007). “Angiotensin receptors, autoimmunity, and preeclampsia.” J Immunol 179(6): 3391-5.
Zybailov, B., M. K. Coleman, et al. (2005). “Correlation of relative abundance ratios derived from peptide ion chromatograms and spectrum counting for quantitative proteomic analysis using stable isotope labeling.” Anal Chem 77(19): 6218-24.
Zybailov, B., A. L. Mosley, et al. (2006). “Statistical analysis of membrane proteome expression changes in Saccharomyces cerevisiae.” J Proteome Res 5(9): 2339-47.

1. Proteomic profile of healthy maternal serum.

2. Proteomic profile of healthy maternal serum in the first trimester of pregnancy.

3. The proteomic profile of claim 2 comprising at least one characteristic expression signature present exclusively during the first trimester of pregnancy.

4. The proteomic profile of claim 3 wherein said characteristic expression signature indicates upregulation of at least one protein selected from the group consisting of chorionic somatomammotropin hormone (P01243), pappalysin-1 (Q13219), pregnancy-specific β-1-glycoprotein 2 (P11465), apolipoprotein C-III (P02656), apolipoprotein A (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), apolipoprotein(a) (P08519), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-1(XVII) chain (Q9UMD9), leucyl-cystinyl aminopeptidase (Q9UIQ6), collagen α-1(I) chain (Q15201), and macrophage colony-stimulating factor (P07333).

5. The proteomic profile of claim 4, wherein said characteristic expression signature indicates upregulation of at least two of said proteins.

6. The proteomic profile of claim 4, wherein said characteristic expression signature indicates upregulation of at least three of said proteins.

7. The proteomic profile of claim 4, wherein said characteristic expression signature indicates upregulation of all of said proteins.

8. Proteomic profile of healthy maternal serum in the second trimester of pregnancy.

9. The proteomic profile of claim 8 comprising at least one characteristic expression signature present exclusively during the second trimester of pregnancy.

10. The proteomic profile of claim 9 wherein said characteristic expression signature indicates upregulation of at least one protein selected from the group consisting of alstrom syndrome protein 1 (Q8TCU4), prolow-density lipoprotein receptor-related protein (Q07954), syndecan-1 (P18827), hypoxia up-regulated protein 1 (Q9Y4L1), dentrix matrix protein 4 (Q81XL6), leucine-rich repeat and calponin homology (Q5VUJ6), plectin-1 (Q15149), and collagen α-2(IX) chain (Q14055).

11. The proteomic profile of claim 10, wherein said characteristic expression signature indicates upregulation of at least two of said proteins.

12. The proteomic profile of claim 10, wherein said characteristic expression signature indicates upregulation of at least three of said proteins.

13. The proteomic profile of claim 10, wherein said characteristic expression signature indicates upregulation of all of said proteins.

14. Proteomic profile of healthy maternal serum in the third trimester of pregnancy.

15. The proteomic profile of claim 14 wherein said characteristic expression signature indicates upregulation of at least one protein selected from the group consisting of pappalysin-1 (Q13219), apolipoprotein C-III (P02656), pregnancy-specific β-1-glycoprotein-1 (P11465), apolipoprotein C-I (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-4(V) chain (P25940), leucyl cystinyl aminopeptidase (Q9UIQ6), collagen α-1(I) chain (Q15201), and macrophage colony-stimulating factor (P07333).

16. The proteomic profile of claim 15, wherein said characteristic expression signature indicates upregulation of at least two of said proteins.

17. The proteomic profile of claim 15, wherein said characteristic expression signature indicates upregulation of at least three of said proteins.

18. The proteomic profile of claim 15, wherein said characteristic expression signature indicates upregulation of all of said proteins.

19. A method for diagnosing a pathologic maternal or fetal condition comprising comparing the proteomic profile of a serum sample obtained from a pregnant human subject to the proteomic profile of maternal serum during healthy gestation of the same gestational age, and determining that said pathologic maternal or fetal condition is present, if there is at least one characteristic expression signature differentiating between the proteomic profiles compared.

20. The method of claim 19, wherein said comparison is implemented using an apparatus adapted to determine the expression of said proteins.

21. The method of claim 19, wherein said comparison is performed by using a software program executed by a suitable processor.

22. The method of claim 21, wherein the program is embodied in software stored on a tangible medium.

23. The method of claim 22 wherein the tangible medium is selected from the group consisting of a flash drive, a CD-ROM, a floppy disk, a hard drive, a DVD, and a memory associated with the processor.

24. The method of any one of claims 19 to 23, further comprising the step of preparing a report recording the results of said testing or the diagnosis.

25. The method of claim 24 wherein said report is recorded or stored on a tangible medium.

26. The method of claim 25 wherein the tangible medium is paper.

27. The method of claim 25 wherein the tangible medium is selected from the group consisting of a flash drive, a CD-ROM, a floppy disk, a hard drive, a DVD, and a memory associated with the processor.

28. The method of any one of claims 19 to 23, further comprising the step of communicating the results of said diagnosis to an interested party.

29. The method of claim 28 wherein the interested party is the patient or the attending physician.

30. The method of claim 28 wherein the communication is in writing, by email, or by telephone.

31. The method of claim 19 wherein the serum sample is obtained in the first trimester of pregnancy.

32. The method of claim 31 wherein said characteristic expression signature indicates upregulation of at least one protein selected from the group consisting of pappalysin-1 (Q13219), apolipoprotein C-III (P02656), apolipoprotein A (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), apolipoprotein(a) (P08519), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-1(XVII) chain (Q9UMD9), leucyl-cystinyl aminopeptidase (Q9UIQ6), collagen α-1(I) chain (Q15201), macrophage colony-stimulating factor (P07333), and pregnancy-specific β-1-glycoprotein 2 (P11465).

33. The method of claim 19 wherein the serum sample is obtained in the second trimester of pregnancy.

34. The method of claim 33 wherein said characteristic expression signature indicates upregulation of at least one protein selected from the group consisting of alstrom syndrome protein 1 (Q8TCU4), prolow-density lipoprotein receptor-related protein (Q07954), syndecan-1 (P18827), hypoxia up-regulated protein 1 (Q9Y4L1), dentrix matrix protein 4 (Q81XL6), leucine-rich repeat and calponin homology (Q5VUJ6), plectin-1 (Q15149), and collagen α-2(IX) chain (Q14055).

35. The method of claim 19 wherein the serum sample is obtained in the third trimester of pregnancy.

36. The method of claim 35 wherein said characteristic expression signature indicates upregulation of at least one protein selected from the group consisting of pappalysin-1 (Q13219), apolipoprotein C-III (P02656), apolipoprotein A (P02564), pregnancy-specific β-1-glycoprotein 1 (Q9P1W5), pregnancy-specific β-1-glycoprotein 9 (Q00887), RNA-binding protein RALY (Q9UKM9), apolipoprotein A-II (P02652), apolipoprotein(a) (P08519), fibulin-1 (Q9UGR4), vascular endothelial growth factor receptor 3 (P35916), ectonucleotide phosphodiesterase (Q13822), nesprin-2 (Q9UJ07), zinc finger protein 512b (Q96KM6), protein FAM40A (Q5VSL9), collagen α-3(V) chain (P25940), cadherin-2 (P19022), collagen α-1(XVII) chain (Q9UMD9), leucyl-cystinyl aminopeptidase (Q9UIQ6), collagen α-1(I) chain (Q15201), macrophage colony-stimulating factor (P07333), and pregnancy-specific β-1-glycoprotein 2 (P11465).

37. A report comprising the results of and/or diagnosis based on a test comprising comparing the proteomic profile of a serum sample obtained from a pregnant human subject to the proteomic profile of maternal serum during healthy gestation of the same gestational age, and determining that said pathologic maternal or fetal condition is present, if there is at least one characteristic expression signature differentiating between the proteomic profiles compared.

38. A tangible medium storing the results of and/or diagnosis based on a test comprising comparing the proteomic profile of a serum sample obtained from a pregnant human subject to the proteomic profile of maternal serum during healthy gestation of the same gestational age, and determining that said pathologic maternal or fetal condition is present, if there is at least one characteristic expression signature differentiating between the proteomic profiles compared.

39. A method for determining the state of maternal or fetal health, comprising comparing a proteomic profile of a test sample of maternal serum obtained from a pregnant female mammalian subject with a proteomic profile of normal maternal serum comprising a unique expression signature wherein the unique expression signature comprises information of the expression of proteins which exhibit continuous upregulation from the first trimester to term.

40. The method of claim 39 wherein the subject is a human patient.

41. The method of claim 40, wherein said comparison is implemented using an apparatus adapted to determine the expression of said proteins.

42. The method of claim 40, wherein said comparison is performed by using a software program executed by a suitable processor.

43. The method of claim 42, wherein the program is embodied in software stored on a tangible medium.

44. The method of claim 43 wherein the tangible medium is selected from the group consisting of a flash drive, a CD-ROM, a floppy disk, a hard drive, a DVD, and a memory associated with the processor.

45. The method of any one of claims 40 to 44, further comprising the step of preparing a report recording the results of said comparison or the diagnosis.

46. The method of claim 45 wherein said report is recorded or stored on a tangible medium.

47. The method of claim 46 wherein the tangible medium is paper.

48. The method of claim 46 wherein the tangible medium is selected from the group consisting of a flash drive, a CD-ROM, a floppy disk, a hard drive, a DVD, and a memory associated with the processor.

49. The method of any one of claims 40 to 44, further comprising the step of communicating the results of said diagnosis to an interested party.

50. The method of claim 49 wherein the interested party is the patient or the attending physician.

51. The method of claim 49 wherein the communication is in writing, by email, or by telephone.

52. The method of claim 39 wherein a deviation from the proteomic profile of normal maternal serum indicates risk of a maternal or a fetal condition.

53. The method of claim 52 wherein said maternal condition is selected from the group consisting of intrauterine infection, preeclampsia, and preterm labor.

54. The method of claim 52 wherein said fetal condition is selected from the group consisting of chromosomal aneuploidies, congenital malformation, fetal infection, gestational age and fetal maturity.

55. The method of claim 54 wherein said chromosomal aneuploidy is selected from the group consisting of Down syndrome, trisomy-13, trisomy-18, Turner syndrome, and Klinefelter syndrome.

56. The method of claim 39 wherein the determination of the state of maternal or fetal health is made during the first trimester.

57. The method of claim 39 wherein the determination of the state of maternal or fetal health is made during the second trimester.

58. The method of claim 39 wherein the determination of the state of maternal or fetal health is made during the third trimester.

59. The method of claim 39 wherein said characteristic expression signature indicates upregulation of two proteins selected from the group consisting of Chorionic somatomammotropin hormone (P01243), Pregnancy-specific beta-1-glycoprotein 1 (P11464), Choriogonadotropin subunit beta (P01233), Pappalysin-1 (Q13219), and Apolipoprotein C-III (P02656).

60. The method of claim 59, wherein said characteristic expression signature indicates upregulation of at least three of said proteins.

61. The method profile of claim 59, wherein said characteristic expression signature indicates upregulation of at least four of said proteins.

62. The method profile of claim 59, wherein said characteristic expression signature indicates upregulation of all of said proteins.

63. A report comprising the results of and/or diagnosis based on a test comprising comparing a proteomic profile of a test sample of maternal serum obtained from a pregnant female mammalian subject with a proteomic profile of normal maternal serum comprising a unique expression signature wherein the unique expression signature comprises information of the expression of proteins which exhibit continuous upregulation from the first trimester to term.

64. A tangible medium storing the results of and/or diagnosis based on a test comprising comparing a proteomic profile of a test sample of maternal serum obtained from a pregnant female mammalian subject with a proteomic profile of normal maternal serum comprising a unique expression signature wherein the unique expression signature comprises information of the expression of proteins which exhibit continuous upregulation from the first trimester to term.

65. A method for determining the state of maternal or fetal health, comprising comparing a proteomic profile of a test sample of maternal serum obtained from a mammalian subject with a proteomic profile of normal maternal serum comprising a unique expression signature wherein the unique expression signature comprises information of the expression of proteins which exhibit continuous down regulation from the first trimester to term.

66. The method of claim 65 wherein the subject is a human patient.

67. The method of claim 66, wherein said comparison is implemented using an apparatus adapted to determine the expression of said proteins.

68. The method of claim 66, wherein said comparison is performed by using a software program executed by a suitable processor.

69. The method of claim 68, wherein the program is embodied in software stored on a tangible medium.

70. The method of claim 69 wherein the tangible medium is selected from the group consisting of a flash drive, a CD-ROM, a floppy disk, a hard drive, a DVD, and a memory associated with the processor.

71. The method of any one of claims 66 to 70, further comprising the step of preparing a report recording the results of said comparison or the diagnosis.

72. The method of claim 71 wherein said report is recorded or stored on a tangible medium.

73. The method of claim 72 wherein the tangible medium is paper.

74. The method of claim 72 wherein the tangible medium is selected from the group consisting of a flash drive, a CD-ROM, a floppy disk, a hard drive, a DVD, and a memory associated with the processor.

75. The method of any one of claims 66 to 70, further comprising the step of communicating the results of said diagnosis to an interested party.

76. The method of claim 75 wherein the interested party is the patient or the attending physician.

77. The method of claim 75 wherein the communication is in writing, by email, or by telephone.

78. The method of claim 65 wherein a deviation from the proteomic profile of normal maternal serum indicates risk of a maternal or a fetal condition.

79. The method of claim 79 wherein said maternal condition is selected from the group consisting of intrauterine infection, preeclampsia, and preterm labor.

80. The method of claim 79 wherein said fetal condition is selected from the group consisting of chromosomal aneuploidies, congenital malformation, fetal infection, gestational age and fetal maturity.

81. The method of claim 80 wherein said chromosomal aneuploidy is selected from the group consisting of Down syndrome, trisomy-13, trisomy-18, Turner syndrome, and Klinefelter syndrome.

82. The method of claim 65 wherein the determination of the state of maternal or fetal health is made during the first trimester.

83. The method of claim 65 wherein the determination of the state of maternal or fetal health is made during the second trimester.

84. The method of claim 65 wherein the determination of the state of maternal or fetal health is made during the third trimester.

85. The method of claim 65 wherein said characteristic expression signature indicates down regulation of two proteins selected from the group consisting of histidine-rich glycoprotein (SEQ ID NO:62), C-reactive protein (SEQ ID NO:68), thrombospondin-1 (SEQ ID NO:60), 14-3-3 protein zelta/delta (SEQ ID NO:61), peroxiredoxin-2 (SEQ ID NO:63), profilin-1 (SEQ ID NO:64), L-selectin (SEQ ID NO:65), ficolin-2 (SEQ ID NO:66), and GDH/6PGL endoplasmic bifunctional protein (SEQ ID NO:67).

86. The method of claim 85, wherein said characteristic expression signature indicates down regulation of at least three of said proteins.

87. The method profile of claim 85, wherein said characteristic expression signature indicates down regulation of at least four of said proteins.

88. The method profile of claim 85, wherein said characteristic expression signature indicates down regulation of all of said proteins.

89. A report comprising the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit continuous down regulation from the first trimester to term.

90. A tangible medium storing the results of and/or diagnosis based on a test comparing a proteomic profile of a test sample of maternal serum obtained from a mammalian subject with a proteomic profile of normal maternal serum comprising a unique expression signature wherein the unique expression signature comprises information of the expression of proteins which exhibit continuous down regulation from the first trimester to term.

91. A method for determining the state of maternal or fetal health, comprising comparing a proteomic profile of a test sample of maternal serum obtained from a mammalian subject with a proteomic profile of normal maternal serum comprising a unique expression signature wherein the unique expression signature comprises information of the expression of proteins which exhibit upregulation from the first trimester to second trimester followed by a slow down until term.

92. The method of claim 91 wherein the subject is a human patient.

93. The method of claim 92, wherein said comparison is implemented using an apparatus adapted to determine the expression of said proteins.

94. The method of claim 92, wherein said comparison is performed by using a software program executed by a suitable processor.

95. The method of claim 94, wherein the program is embodied in software stored on a tangible medium.

96. The method of claim 95 wherein the tangible medium is selected from the group consisting of a flash drive, a CD-ROM, a floppy disk, a hard drive, a DVD, and a memory associated with the processor.

97. The method of any one of claims 92 to 96, further comprising the step of preparing a report recording the results of said comparison or the diagnosis.

98. The method of claim 98 wherein said report is recorded or stored on a tangible medium.

99. The method of claim 98 wherein the tangible medium is paper.

100. The method of claim 98 wherein the tangible medium is selected from the group consisting of a flash drive, a CD-ROM, a floppy disk, a hard drive, a DVD, and a memory associated with the processor.

101. The method of any one of claims 92 to 96, further comprising the step of communicating the results of said diagnosis to an interested party.

102. The method of claim 101 wherein the interested party is the patient or the attending physician.

103. The method of claim 101 wherein the communication is in writing, by email, or by telephone.

104. The method of claim 91 wherein a deviation from the proteomic profile of normal maternal serum indicates risk of a maternal or a fetal condition.

105. The method of claim 104 wherein said maternal condition is selected from the group consisting of intrauterine infection, preeclampsia, and preterm labor.

106. The method of claim 104 wherein said fetal condition is selected from the group consisting of chromosomal aneuploidies, congenital malformation, fetal infection, gestational age and fetal maturity.

107. The method of claim 106 wherein said chromosomal aneuploidy is selected from the group consisting of Down syndrome, trisomy-13, trisomy-18, Turner syndrome, and Klinefelter syndrome.

108. The method of claim 91 wherein the determination of the state of maternal or fetal health is made during the first trimester.

109. The method of claim 91 wherein the determination of the state of maternal or fetal health is made during the second trimester.

110. The method of claim 91 wherein the determination of the state of maternal or fetal health is made during the third trimester.

111. The method of claim 91 wherein said characteristic expression signature indicates upregulation from the first trimester to second trimester followed by a slow down until term of at least one protein selected from the group consisting of pregnancy zone protein (SEQ ID NO: 18), corticosteroid-binding globulin (SEQ ID NO:27), and bone-marrow proteoglycan 2 (SEQ ID NO:16).

112. The method of claim 111, wherein said characteristic expression signature indicates upregulation from the first trimester to second trimester followed by a slow down until term of at least two of said proteins.

113. The method profile of claim 111, wherein said characteristic expression signature indicates upregulation from the first trimester to second trimester followed by a slow down until term of all of said proteins.

114. A report comprising the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit upregulation from the first trimester to second trimester followed by a slow down until term.

115. A tangible medium storing the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit upregulation from the first trimester to second trimester followed by a slow down until term.

116. A method for determining the state of maternal or fetal health, comprising comparing a proteomic profile of a test sample of maternal serum obtained from a mammalian subject with a proteomic profile of normal maternal serum comprising a unique expression signature wherein the unique expression signature comprises information of the expression of proteins which exhibit down regulation from the first trimester to second trimester followed by a slow down until term.

117. The method of claim 116 wherein the subject is a human patient.

118. The method of claim 117, wherein said comparison is implemented using an apparatus adapted to determine the expression of said proteins.

119. The method of claim 117, wherein said comparison is performed by using a software program executed by a suitable processor.

120. The method of claim 119, wherein the program is embodied in software stored on a tangible medium.

121. The method of claim 120 wherein the tangible medium is selected from the group consisting of a flash drive, a CD-ROM, a floppy disk, a hard drive, a DVD, and a memory associated with the processor.

122. The method of any one of claims 117 to 121, further comprising the step of preparing a report recording the results of said comparison or the diagnosis.

123. The method of claim 122 wherein said report is recorded or stored on a tangible medium.

124. The method of claim 123 wherein the tangible medium is paper.

125. The method of claim 123 wherein the tangible medium is selected from the group consisting of a flash drive, a CD-ROM, a floppy disk, a hard drive, a DVD, and a memory associated with the processor.

126. The method of any one of claims 117 to 121, further comprising the step of communicating the results of said diagnosis to an interested party.

127. The method of claim 126 wherein the interested party is the patient or the attending physician.

128. The method of claim 126 wherein the communication is in writing, by email, or by telephone.

129. The method of claim 116 wherein a deviation from the proteomic profile of normal maternal serum indicates risk of a maternal or a fetal condition.

130. The method of claim 129 wherein said maternal condition is selected from the group consisting of placental pathology, intrauterine infection, preeclampsia, and preterm labor.

131. The method of claim 129 wherein said fetal condition is selected from the group consisting of chromosomal aneuploidies, congenital malformation, fetal infection, gestational age and fetal maturity.

132. The method of claim 131 wherein said chromosomal aneuploidy is selected from the group consisting of Down syndrome, trisomy-13, trisomy-18, Turner syndrome, and Klinefelter syndrome.

133. The method of claim 116 wherein the determination of the state of maternal or fetal health is made during the first trimester.

134. The method of claim 116 wherein the determination of the state of maternal or fetal health is made during the second trimester.

135. The method of claim 116 wherein the determination of the state of maternal or fetal health is made during the third trimester.

136. The method of claim 116 wherein said characteristic expression signature indicates down regulation from the first trimester to second trimester followed by a slow down until term of human choriogonadotropin subunit β (SEQ ID NO:29).

137. A report comprising the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit down regulation from the first trimester to second trimester followed by a slow down until term.

138. A tangible medium storing the results of and/or diagnosis based on a test comprising comparing a proteomic profile of a test sample of maternal serum obtained from a mammalian subject with a proteomic profile of normal maternal serum comprising a unique expression signature wherein the unique expression signature comprises information of the expression of proteins which exhibit down regulation from the first trimester to second trimester followed by a slow down until term.

139. An immunoassay kit comprising antibodies and reagents for the detection of two or more proteins selected from the group consisting of chorionic somatomammotropin hormone (P01243), Pregnancy-specific beta-1-glycoprotein 1 (P11464), Choriogonadotropin subunit beta (P01233), Pappalysin-1 (Q13219), and Apolipoprotein C-III (P02656).

140. A proteomic profile of healthy maternal serum from a pregnant subject, wherein the pregnancy resulted from in vitro fertilization.

141. A method for determining the state of placental health, comprising comparing a proteomic profile of a test sample of maternal serum obtained from a mammalian subject whose pregnancy resulted from in vitro fertilization with the proteomic profile of a normal sample.

142. A method for predicting small for gestational age comprising comparing the proteomic profile of a serum sample obtained from a pregnant human subject to the proteomic profile of maternal serum during healthy gestation of the same gestational age, and determining that said small for gestational age is more likely than not to be present, if there is at least one characteristic expression signature differentiating between the proteomic profiles compared.

143. A method for predicting fetal loss comprising comparing the proteomic profile of a serum sample obtained from a pregnant human subject to the proteomic profile of maternal serum during healthy gestation of the same gestational age, and determining that said fetal loss is more likely than not to occur, if there is at least one characteristic expression signature differentiating between the proteomic profiles compared.


SEQUENCE LISTING (69 sequences)

SEQ ID NO: 1 - protein, 1628 aa, Homo sapiens
MRLWSWVLHLGLLSAALGCGLAERPRRARRDPRAGRPPRPAAGPATCATRAARGRRASPPPPPPPGGAWEAVRVPRRRQQREARGATEEPSPPSRALYFSGRGEQLRVLRADLELPRDAFTLQVWLRAEGGQRSPAVITGLYDKCSYISRDRGWVVGIHTISDQDNKDPRYFFSLKTDRARQVTTINAHRSYLPGQWVYLAATYDGQFMKLYVNGAQVATSGEQVGGIFSPLTQKCKVLMLGGSALNHNYRGYIEHFSLWKVARTQREILSDMETHGAHTALPQLLLQENWDNVKHAWSPMKDGSSPKVEFSNAHGFLLDTSLEPPLCGQTLCDNTEVIASYNQLSSFRQPKVVRYRVVNLYEDDHKNPTVTREQVDFQHHQLAEAFKQYNISWELDVLEVSNSSLRRRLILANCDISKIGDENCDPECNHTLTGHDGGDCRHLRHPAFVKKQHNGVCDMDCNYERFNFDGGECCDPEITNVTQTCFDPDSPHRAYLDVNELKNILKLDGSTHLNIFFAKSSEEELAGVATWPWDKEALMHLGGIVLNPSFYGMPGHTHTMIHEIGHSLGLYHVFRGISEIQSCSDPCMETEPSFETGDLCNDTNPAPKHKSCGDPGPGNDTCGFHSFFNTPYNNFMSYADDDCTDSFTPNQVARMHCYLDLVYQGWQPSRKPAPVALAPQVLGHTTDSVTLEWFPPIDGHFFERELGSACHLCLEGRILVQYASNASSPMPCSPSGHWSPREAEGHPDVEQPCKSSVRTWSPNSAVNPHTVPPACPEPQGCYLELEFLYPLVPESLTIWVTFVSTDWDSSGAVNDIKLLAVSGKNISLGPQNVFCDVPLTIRLWDVGEEVYGIQIYTLDEHLEIDAAMLTSTADTPLCLQCKPLKYKVVRDPPLQMDVASILHLNRKFVDMDLNLGSVYQYWVITISGTEESEPSPAVTYIHGRGYCGDGIIQKDQGEQCDDMNKINGDGCSLFCRQEVSFNCIDEPSRCYFHDGDGVCEEFEQKTSIKDCGVYTPQGFLDQWASNASVSHQDQQCPGWVIIGQPAASQVCRTKVIDLSEGISQHAWYPCTISYPYSQLAQTTFWLRAYFSQPMVAAAVIVHLVTDGTYYGDQKQETISVQLLDTKDQSHDLGLHVLSCRNNPLIIPVVHDLSQPFYHSQAVRVSFSSPLVAISGVALRSFDNFDPVTLSSCQRGETYSPAEQSCVHFACEKTDCPELAVENASLNCSSSDRYHGAQCTVSCRTGYVLQIRRDDELIKSQTGPSVTVTCTEGKWNKQVACEPVDCSIPDHHQVYAASFSCPEGTTFGSQCSFQCRHPAQLKGNNSLLTCMEDGLWSFPEALCELMCLAPPPVPNADLQTARCRENKHKVGSFCKYKCKPGYHVPGSSRKSKKRAFKTQCTQDGSWQEGACVPVTCDPPPPKFHGLYQCTNGFQFNSECRIKCEDSDASQGLGSNVIHCRKDGTWNGSFHVCQEMQGQCSVPNELNSNLKLQCPDGYAIGSECATSCLDHNSESIILPMNVTVRDIPHWLNPTRVERVVCTAGLKWYPHPALIHCVKGCEPFMGDNYCDAINNRAFCNYDGGDCCTSTVKTKKVTPFPMSCDLQGDCACRDPQAQEHSRKDLRGYSHG

SEQ ID NO: 2 - protein, 1025 aa, Homo sapiens
MEPFTNDRLQLPRNMIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 3 - protein, 909 aa, Homo sapiens
MAARPLPVSPARALLLALAGALLAPCEARGVSLWNQGRADEVVSASVRSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 4 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 5 - protein, 99 aa, Homo sapiens
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 6 - protein, 217 aa, Homo sapiens
MAPGSRTSLLLAFALLCLPWLQEAGAVQTVPLSRLFDHAMLQAHRAHQLAIDTYQEFEETYIPKDQKYSFLHDSQTSFCFSDSIPTPSNMEETQQKSNLELLRISLLLIESWLEPVRFLRSMFANNLVYDTSDSDDYHLLKDLEEGIQTLMGRLEDGSRRTGQILKQTYSKFDTNSHNHDALLKNYGLLYCFRKDMDKVETFLRMVQCRSVEGSCGF

SEQ ID NO: 7 - protein, 1685 aa, Homo sapiens
MKLRGVSLAAGLFLLALSLWGQPAEAAACYGCSPGSKCDCSGIKGEKGERGFPGLEGHPGLPGFPGPEGPPGPRGQKGDDGIPGPPGPKGIRGPPGLPGFPGTPGLPGMPGHDGAPGPQGIPGCNGTKGERGFPGSPGFPGLQGPPGPPGIPGMKGEPGSIIMSSLPGPKGNPGYPGPPGIQGLPGPTGIPGPIGPPGPPGLMGPPGPPGLPGPKGNMGLNFQGPKGEKGEQGLQGPPGPPGQISEQKRPIDVEFQKGDQGLPGDRGPPGPPGIRGPPGPPGGEKGEKGEQGEPGKRGKPGKDGENGQPGIPGLPGDPGYPGEPGRDGEKGQKGDTGPPGPPGLVIPRPGTGITIGEKGNIGLPGLPGEKGERGFPGIQGPPGLPGPPGAAVMGPPGPPGFPGERGQKGDEGPPGISIPGPPGLDGQPGAPGLPGPPGPAGPHIPPSDEICEPGPPGPPGSPGDKGLQGEQGVKGDKGDTCFNCIGTGISGPPGQPGLPGLPGPPGSLGFPGQKGEKGQAGATGPKGLPGIPGAPGAPGFPGSKGEPGDILTFPGMKGDKGELGSPGAPGLPGLPGTPGQDGLPGLPGPKGEPGGITFKGERGPPGNPGLPGLPGNIGPMGPPGFGPPGPVGEKGIQGVAGNPGQPGIPGPKGDPGQTITQPGKPGLPGNPGRDGDVGLPGDPGLPGQPGLPGIPGSKGEPGIPGIGLPGPPGPKGFPGIPGPPGAPGTPGRIGLEGPPGPPGFPGPKGEPGFALPGPPGPPGLPGFKGALGPKGDRGFPGPPGPPGRTGLDGLPGPKGDVGPNGQPGPMGPPGLPGIGVQGPPGPPGIPGPIGQPGLHGIPGEKGDPGPPGLDVPGPPGERGSPGIPGAPGPIGPPGSPGLPGKAGASGFPGTKGEMGMMGPPGPPGPLGIPGRSGVPGLKGDDGLQGQPGLPGPTGEKGSKGEPGLPGPPGPMDPNLLGSKGEKGEPGLPGIPGVSGPKGYQGLPGDPGQPGLSGQPGLPGPPGPKGNPGLPGQPGLIGPPGLKGTIGDMGFPGPQGVEGPPGPSGVPGQPGSPGLPGQKGDKGDPGISSIGLPGLPGPKGEPGLPGYPGNPGIKGSVGDPGLPGLPGTPGAKGQPGLPGFPGTPGPPGPKGISGPPGNPGLPGEPGPVGGGGHPGQPGPPGEKGKPGQDGIPGPAGQKGEPGQPGFGNPGPPGLPGLSGQKGDGGLPGIPGNPGLPGPKGEPGFHGFPGVQGPPGPPGSPGPALEGPKGNPGPQGPPGRPGLPGPEGPPGLPGNGGIKGEKGNPGQPGLPGLPGLKGDQGPPGLQGNPGRPGLNGMKGDPGLPGVPGFPGMKGPSGVPGSAGPEGEPGLIGPPGPPGLPGPSGQSIIIKGDAGPPGIPGQPGLKGLPGPQGPQGLPGPTGPPGDPGRNGLPGFDGAGGRKGDPGLPGQPGTRGLDGPPGPDGLQGPPGPPGTSSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 8 - protein, 419 aa, Homo sapiens
MGTLSAPPCTQRIKWKGLLLTASLLNFWNLPTTAQVTIEAEPTKVSEGKDVLLLVHNLPQNLTGYIWYKGQMRDLYHYITSYVVDGEIIIYGPAYSGRETAYSNASLLIQNVTREDAGSYTLHIIKGDDGTRGVTGRFTFTLHLETPKPSISSSNLNPRETMEAVSLTCDPETPDASYLWWMNGQSLPMTHSLKLSETNRTLFLLGVTKYTAGPYECEIRNPVSASRSDPVTLNLLPKLPKPYITINNLNPRENKDVLNFTCEPKSENYTYIWWLNGQSLPVSPRVKRPIENRILILPSVTRNETGPYQCEIRDRYGGIRSDPVTLNVLYGPDLPRIYPSFTYYRSGEVLYLSCSADSNPPAQYSWTINEKFQLPGQKLFIRHITTKHSGLYVCSVRNSATGKESSKSMTVEVSDWTVP

SEQ ID NO: 9 - protein, 335 aa, Homo sapiens
MGPLSAPPCTEHIKWKGLLVTASLLNFWNLPTTAQVTIEAQPPKVSEGKDVLLLVHNLPQNLTGYIWYKGQIRDLYHYITSYVVDGQIIIYGPAYSGRETAYSNASLLIQNVTREDAGSYTLHIIKRGDGTRGVTGYFTFTLYLETPKPSISSSNLNPREAMETVILTCDPETPDTSYQWWMNGQSLPMTHRFQLSETNRTLFLFGVTKYTAGPYECEIRNSGSASRSDPVTLNLLHGPDLPRIHPSYTNYRSGDNLYLSCFANSNPPAQYSWTINGKFQQSGQNLFIPQITTKHSGLYVCSVRNSATGEESSTSLTVKVSASTRIGLLPLLNPT

SEQ ID NO: 10 - protein, 419 aa, Homo sapiens [sequence omitted]

SEQ ID NO: 11 - protein, 419 aa, Homo sapiens
MGPLSAPPCTQHITWKGLLLTASLLNFWNPPTTAQVTIEAQPPKVSEGKDVLLLVHNLPQNLTGHIWYKGQIRDLYHYVTSYIVDGQIIKYGPAYSGRETVYSNASLLIQNVTQEDTGSYTLHIIKRGDGTGGVTGRFTFTLYLETPKPSISSSNFNPREATEAVILTCDPETPDASYLWWMNGQSLPMTHSLQLSETNRTLYLFGVTNYTAGPYECEIRNPVSASRSDPVTLNLLPKLPKPYITINNLNPRENKDVSTFTCEPKSENYTYIWWLNGQSLPVSPRVKRRIENRILILPSVTRNETGPYQCEIRDRYGGIRSDPVTLNVLYGPDLPRIYPSFTYYHSGQNLYLSCFADSNPPAQYSWTINGKFQLSGQKLSIPQITTKHSGLYACSVRNSATGKESSKSVTVRVSDWTLP

SEQ ID NO: 12 - protein, 426 aa, Homo sapiens
MGPLPAPSCTQRITWKGLLLTASLLNFWNPPTTAEVTIEAQPPKVSEGKDVLLLVHNLPQNLPGYFWYKGEMTDLYHYIISYIVDGKIIIYGPAYSGRETVYSNASLLIQNVTRKDAGTYTLHIIKRGDETREEIRHFTFTLYLETPKPYISSSNLNPREAMEAVRLICDPETLDASYLWWMNGQSLPVTHRLQLSKTNRTLYLFGVTKYIAGPYECEIRNPVSASRSDPVTLNLLPKLPIPYITINNLNPRENKDVLAFTCEPKSENYTYIWWLNGQSLPVSPGVKRPIENRILILPSVTRNETGPYQCEIRDRYGGLRSNPVILNVLYGPDLPRIYPSFTYYRSGENLDLSCFTESNPPAEYFWTINGKFQQSGQKLFIPQITRNHSGLYACSVHNSATGKEISKSMTVKVSGPCHGDLTESQS

SEQ ID NO: 13 - protein, 481 aa, Homo sapiens
MGALARALPSILLALLLTSTPEALGANPGLVARITDKGLQYAAQEGLLALQSELLRITLPDFTGDLRIPHVGRGRYEFHSLNIHSCELLHSALRPVPGQGLSLSISDSSIRVQGRWKVRKSFFKLQGSFDVSVKGISISVNLLLGSESSGRPTVTASSCSSDIADVEVDMSGDLGWLLNLFHNQIESKFQKVLESRICEMIQKSVSSDLQPYLQTLPVTTEIDSFADIDYSLVEAPRATAQMLEVMFKGEIFHRNHRSPVTLLAAVMSLPEEHNKMVYFAISDYVFNTASLVYHEEGYLNFSITDDMIPPDSNIRLTTKSFRPFVPRLARLYPNMNLELQGSVPSAPLLNFSPGNLSVDPYMEIDAFVLLPSSSKEPVFRLSVATNVSATLTFNTSKITGFLKPGKVKVELKESKVGLFNAELLEALLNYYILNTFYPKFNDKLAEGFPLPLLKRVQLYDLGLQIHKDFLFLGANVQYMRV

SEQ ID NO: 14 - protein, 703 aa, Homo sapiens
MERAAPSRRVPLPLLLLGGLALLAAGVDADVLLEACCADGHRMATHQKDCSLPYATESKECRMVQEQCCHSQLEELHCATGISLANEQDRCATPHGDNASLEATFVKRCCHCCLLGRAAQAQGQSCEYSLMVGYQCGQVFRACCVKSQETGDLDVGGLQETDKIIEVEEEQEDPYLNDRCRGGGPCKQQCRDTGDEVVCSCFVGYQLLSDGVSCEDVNECITGSHSCRLGESCINTVGSFRCQRDSSCGTGYELTEDNSCKDIDECESGIHNCLPDFICQNTLGSFRCRPKLQCKSGFIQDALGNCIDINECLSISAPCPIGHTCINTEGSYTCQKNVPNCGRGYHLNEEGTRCVDVDECAPPAEPCGKGHRCVNSPGSFRCECKTGYYFDGISRMCVDVNECQRYPGRLCGHKCENTLGSYLCSCSVGFRLSVDGRSCEDINECSSSPCSQECANVYGSYQCYCRRGYQLSDVDGVTCEDIDECALPTGGHICSYRCINIPGSFQCSCPSSGYRLAPNGRNCQDIDECVTGIHNCSINETCFNIQGGFRCLAFECPENYRRSAATLQQEKTDTVRCIKSCRPNDVTCVFDPVHTISHTVISLPTFREFTRPEEIIFLRAITPPHPASQANIIFDITEGNLRDSFDIIKRYMDGMTVGVVRQVRPIVGPFHAVLKLEMNYVVGGVVSHRNVVNVRIFVSEYWF

SEQ ID NO: 15 - protein, 101 aa, Homo sapiens
MGTRLLPALFLVLLVLGFEVQGTQQPQQDEMPSPTFLTQVKESLSSYWESAKTAAQNLYEKTYLPAVDEKLRDLYSKSTAAMSTYTGIFTDQVLSVLKGEE

SEQ ID NO: 16 - protein, 222 aa, Homo sapiens
MKLPLLLALLFGAVSALHLRSETSTFETPLGAKTLPEDEETPEQEMEETPCRELEEEEEWGSGSEDASKKDGAVESISVPDMVDKNLTCPEEEDTVKVVGIPGCQTCRYLLVRSLQTFSQAWFTCRRCYRGNLVSIHNFNINYRIQCSVSALNQGQVWIGGRITGSGRCRRFQWVDGSRWNFAYWAAHQPWSRGGHCVALCTRGGYWRRAHCLRRLPFICSY

SEQ ID NO: 17 - protein, 866 aa, Homo sapiens
MFSMRIVCLVLSVVGTAWTADSGEGDFLAEGGGVRGPRVVERHQSACKDSDWPFCSDEDWNYKCPSGCRMKGLIDEVNQDFTNRINKLKNSLFEYQKNNKDSHSLTTNIMEILRGDFSSANNRDNTYNRVSEDLRSRIEVLKRKVIEKVQHIQLLQKNVRAQLVDMKRLEVDIDIKIRSCRGSCSRALAREVDLKDYEDQQKQLEQVIAKDLLPSRDRQHLPLIKMKPVPDLVPGNFKSQLQKVPPEWKALTDMPQMRMELERPGGNEITRGGSTSYGTGSETESPRNPSSAGSWNSGSSGPGSTGNRNPGSSGTGGTATWKPGSSGPGSTGSWNSGSSGTGSTGNQNPGSPRPGSTGTWNPGSSERGSAGHWTSESSVSGSTGQWHSESGSFRPDSPGSGNARPNNPDWGTFEEVSGNVSPGTRREYHTEKLVTSKGDKELRTGKEKVTSGSTTTTRRSCSKTVTKTVIGPDGHKEVTKEVVTSEDGSDCPEAMDLGTLSGIGTLDGFRHRHPDEAAFFDTASTGKTFPGFFSPMLGEFVSETESRGSESGIFTNTKESSSHHPGIAEFPSRGKSSSYSKQFTSSTSYNRGDSTFESKSYKMADEAGSEADHEGTHSTKRGHAKSRPVRDCDDVLQTHPSGTQSGIFNIKLPGSSKIFSVYCDQETSLGGWLLIQQRMDGSLNFNRTWQDYKRGFGSLNDEGEGEFWLGNDYLHLLTQRGSVLRVELEDWAGNEAYAEYHFRVGSEAEGYALQVSSYEGTAGDALIEGSVEEGAEYTSHNNMQFSTFDRDADQWEENCAEVYGGGWWYNNCQAANLNGIYYPGGSYDPRNNSPYEIENGVVWVSFRGADYSLRAVRMKIRPLVTQ

SEQ ID NO: 18 - protein, 1482 aa, Homo sapiens
MRKDRLLHLCLVLLLILLSASDSNSTEPQYMVLVPSLLHTEAPKKGCVLLSHLNETVTVSASLESGRENRSLFTDLVAEKDLFHCVSFTLPRISASSEVAFLSIQIKGPTQDFRKRNTVLVLNTQSLVFVQTDKPMYKPGQTVRFRVVSVDENFRPRNELIPLIYLENPRRNRIAQWQSLKLEAGINQLSFPLSSEPIQGSYRVVVQTESGGRIQHPFTVEEFVLPKFEVKVQVPKIISIMDEKVNITVCGEYTYGKPVPGLATVSLCRKLSRVLNCDKQEVCEEFSQQLNSNGCITQQVHTKMLQITNTGFEMKLRVEARIREEGTDLEVTANRISEITNIVSKLKFVKVDSHFRQGIPFFAQVLLVDGKGVPIPNKLFFISVNDANYYSNATTNEQGLAQFSINTTSISVNKLFVRVFTVHPNLCFHYSWVAEDHQGAQHTANRVFSLSGSYIHLEPVAGTLPCGHTETITAHYTLNRQAMGELSELSFHYLIMAKGVIVRSGTHTLPVESGDMKGSFALSFPVESDVAPIARMFIFAILPDGEVVGDSEKFEIENCLANKVDLSFSPAQSPPASHAHLQVAAAPQSLCALRAVDQSVLLMKPEAELSVSSVYNLLTVKDLTNFPDNVDQQEEEQGHCPRPFFIHNGAIYVPLSSNEADIYSFLKGMGLKVFTNSKIRKPKSCSVIPSVSAGAVGQGYYGAGLGVVERPYVPQLGTYNVIPLNNEQSSGPVPETVRSYFPETWIWELVAVNSSGVAEVGVTVPDTITEWKAGAFCLSEDAGLGISSTASLRAFQPFFVELTMPYSVIRGEVFTLKATVLNYLPKCIRVSVQLKASPAFLASQNTKGEESYCICGSERQTLSWTVTPKTLGNVNFSVSAEAMQSLELCGNEVVEVPEIKRKDTVIKTLLVEAEGIEQEKTFSSMTCASGANVSEQLSLKLPSNVVKESARASFSVLGDILGSAMQNIQNLLQMPYGCGEQNMVLFAPNIYVLNYLNETQQLTQEIKAKAVGYLITGYQRQLNYKHQDGSYSTFGERYGRNQGNTWLTAFVLKTFAQARSYIFIDEAHITQSLTWLSQMQKDNGCFRSSGSLLNNAIKGGVEDEATLSAYVTIALLEIPLPVTNPIVRNALFCLESAWNVAKEGTHGSHVYTKALLAYAFSLLGKQNQNREILNSLDKEAVKEDNLVHWERPQRPKAPVGHLYQTQAPSAEVEMTSYVLLAYLTAQPAPTSGDLTSATNIVKWIMKQQNAQGGFSSTQDTVVALHALSRYGAATFTRTEKTAQVTVQDSQTFSTNFQVDNNNLLLLQQISLPELPGEYVITVTGERCVYLQTSMKYNILPEKEDSPFALKVQTVPQTCDGHKAHTSFQISLTISYTGNRPASNMVIVDVKMVSGFIPLKPTVKMLERSSSVSRTEVSNNHVLIYVEQVTNQTLSFSFMVLQDIPVGDLKPAIVKVYDYYETDESVVAEYIAPCSTDTEHGNV

SEQ ID NO: 19 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 20 - protein, 863 aa, Homo sapiens [sequence omitted]

SEQ ID NO: 21 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 22 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSKLEDNIRRLRALADGVQKVHKGTTIANVVSGSLSISSGILTLVGMGLAPFTEGGSLVLLEPGMELGITAALTGITSSTIDYGKKWWTQAQAHDLVIKSLDKLKEVKEFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 23 - protein, 1075 aa, Homo sapiens
MRGGKCNMLSSLGCLLLCGSITLALGNAQKLPKGKRPNLKVHINTTSDSILLKFLRPSPNVKLEGLLLGYGSNVSPNQYFPLPAEGKFTEAIVDAEPKYLIVVRPAPPPSQKKSCSGKTRSRKPLQLVVGTLTPSSVFLSWGFLINPHHDWTLPSHCPNDRFYTIRYREKDKEKKWIFQICPATETIVENLKPNTVYEFGVKDNVEGGIWSKIFNHKTVVGSKKVNGKIQSTYDQDHTVPAYVPRKLIPITIIKQVIQNVTHKDSAKSPEKAPLGGVILVHLIIPGLNETTVKLPASLMFEISDALKTQLAKNETLALPAESKTPEVEKISARPTTVTPETVPRSTKPTTSSALDVSETTLASSEKPWIVPTAKISEDSKVLQPQTATYDVFSSPTTSDEPEISDSYTATSDRILDSIPPKTSRTLEQPRATLAPSETPFVPQKLEIFTSPEMQPTTPAPQQTTSIPSTPKRRPRPKPPRTKPERTTSAGTITPKISKSPEPTWTTPAPGKTQFISLKPKIPLSPEVTHTKPAPKQTPRAPPKPKTSPRPRIPQTQPVPKVPQRVTAKPKTSPSPEVSYTTPAPKDVLLPHKPYPEVSQSEPAPLETRGIPFIPMISPSPSQEELQTTLEETDQSTQEPFTTKIPRTTELAKTTQAPHRFYTTVRPRTSDKPHIRPGVKQAPRPSGADRNVSVDSTHPTKKPGTRRPPLPPRPTHPRRKPLPPNNVTGKPGSAGIISSGPITTPPLRSTPRPTGTPLERIETDIKQPTVPASGEELENITDFSSSPTRETDPLGKPRFKGPHVRYIQKPDNSPCSITDSVKRFPKEEATEGNATSPPQNPPTNLTVVTVEGCPSFVILDWEKPLNDTVTEYEVISRENGSFSGKNKSIQMTNQTFSTVENLKPNTSYEFQVKPKNPLGEGPVSNTVAFSTESADPRVSEPVSAGRDAIWTERPFNSDSYSECKGKQYVKRTWYKKFVGVQLCNSLRYKIYLSDSLTGKFYNIGDQRGHGEDHCQFVDSFLDGRTGQQLTSDQLPIKEGYFRAVRQEPVQFGEIGGHTQINYVQWYECGTTIPGKW

SEQ ID NO: 24 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 25 - protein, 317 aa, Homo sapiens [sequence omitted]

SEQ ID NO: 26 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 27 - protein, 405 aa, Homo sapiens
MPLLLYTCLLWLPTSGLWTVQAMDPNAAYVNMSNHHRGLASANVDFAFSLYKHLVALSPKKNIFISPVSISMALAMLSLGTCGHTRAQLLQGLGFNLTERSETEIHQGFQHLHQLFAKSDTSLEMTMGNALFLDGSLELLESFSADIKHYYESEVLAMNFQDWATASRQINSYVKNKTQGKIVDLFSGLDSPAILVLVNYIFFKGTWTQPFDLASTREENFYVDETTVVKVPMMLQSSTISYLHDSELPCQLVQMNYVGNGTVFFILPDKGKMNTVIAALSRDTINRWSAGLTSSQVDLYIPKVTISGVYDLGDVLEEMGIADLFTNQANFSRITQDAQLKSSKVVHKAVLQLNEEGVDTAGSTGVTLNLTSKPIILRFNQPFIIMIFDHFTWSSLFLARVMNPV

SEQ ID NO: 28 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKTSKNTRYTHGHGSETLYLAPGGGDDWIYDLGIKYSFTIELRDTGTYGFLLPERYIKPTCREAFAAVSKIAWHVIRNV

SEQ ID NO: 29 - protein, 165 aa, Homo sapiens
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 30 - protein, 299 aa, Homo sapiens
MDLLWILPSLWLLLLGGPACLKTQEHPSCPGPRELEASKVVLLPSCPGAPGSPGEKGAPGPQGPPGPPGKMGPKGEPGDPVNLLRCQEGPRNCRELLSQGATLSGWYHLCLPEGRALPVFCDMDTEGGGWLVFQRRQDGSVDFFRSWSSYRAGFGNQESEFWLGNENLHQLTLQGNWELRVELEDFNGNRTFAHYATFRLLGEVDHYQLALGKFSEGTAGDSLSLHSGRPFTTYDADHDSSNSNCAVIVHGAWWYASCYRSNLNGRYAVSEAAAHKYGIDWASGRGVGHPYRRVRMMLR

SEQ ID NO: 31 - protein, 1427 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 32 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 33 - protein, 711 aa, Homo sapiens
MGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKDYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREFCDLPRCGSEAQPRQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVALLNVISNQECNIKHRGRVRESEMCTEGLLAPVGACEGDYGGPLACFTHNCWVLEGIIIPNRVCARSRWPAVFTRVSVFVDWIHKVMRLG

SEQ ID NO: 34 - protein, 2386 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTQGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 35 - protein, 840 aa, Homo sapiens
MSAFRLWPGLLIMLGSLCHRGSPCGLSTHVEIGHRALEFLQLHNGRVNYRELLLEHQDAYQAGIVFPDCFYPSICKGGKFHDVSESTHWTPFLNASVHYIRENYPLPWEKDTEKLVAFLFGITSHMAADVSWHSLGLEQGFLRTMGAIDFHGSYSEAHSAGDFGGDVLSQFEFNFNYLARRWYVPVKDLLGIYEKLYGRKVITENVIVDCSHIQFLEMYGEMLAVSKLYPTYSTKSPFLVEQFQEYFLGGLDDMAFWSTNIYHLTSFMLENGTSDCNLPENPLFIACGGQQNHTQGSKMQKNDFHRNLTTSLTESVDRNINYTERGVFFSVNSWTPDSMSFIYKALERNIRTMFIGGSQLSQKHVSSPLASYFLSFPYARLGWAMTSADLNQDGHGDLVVGAPGYSRPGHIHIGRVYLIYGNDLGLPPVDLDLDKEAHRILEGFQPSGRFGSALAVLDFNVDGVPDLAVGAPSVGSEQLTYKGAVYVYFGSKQGGMSSSPNITISCQDIYCNLGWTLLAADVNGDSEPDLVIGSPFAPGGGKQKGIVAAFYSGPSLSDKEKLNVEAANWTVRGEEDFSWFGYSLHGVTVDNRTLLLVGSPTWKNASRLGHLLHIRDEKKSLGRVYGYFPPNGQSWFTISGDKAMGKLGTSLSSGHVLMNGTLKQVLLVGAPTYDDVSKVAFLTVTLHQGGATRMYALTSDAQPLLLSTFSGDRRFSRFGGVLHLSDLDDDGLDEIIMAAPLRIADVTSGLIGGEDGRVYVYNGKETTLGDMTGKCKSWITPCPEEKAQYVLISPEASSRFGSSLITVRSKAKNQVVIAAGRSSLGARLSGALHVYSLGSD

SEQ ID NO: 36 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 37 - protein, 458 aa, Homo sapiens
MSDLLSVFLHLLLLFKLVAPVTFRHHRYDDLVRTLYKVQNECPGITRVYSIGRSVEGRHLYVLEFSDHPGIHEPLEPEVKYVGNMHGNEALGRELMLQLSEFLCEEFRNRNQRIVQLIQDTRIHILPSMNPDGYEVAAAQGPNKPGYLVGRNNANGVDLNRNFPDLNTYIYYNEKYGGPNHHLPLPDNWKSQVEPETRAVIRWMHSFNFVLSANLHGGAVVANYPYDKSFEHRVRGVRRTASTPTPDDKLFQKLAKVYSYAHGWMFQGWNCGDYFPDGITNGASWYSLSKGMQDFNYLHTNCFEITLELSCDKFPPEEELQREWLGNREALIQFLEQVHQGIKGMVLDENYNNLANAVISVSGINHDVTSGDHGDYFRLLLPGIYTVSATAPGYDPETVTVTVGPAEPTLVNFHLKRSIPQVSPVRRAPSRRHGVRAKVQPQARKKEMEMRQLQRGPA

SEQ ID NO: 38 - protein, 655 aa, Homo sapiens
MGRWAWVPSPWPPPGLGPFLLLLLLLLLLPRGFQPQPGGNRTESPEPNATATPAIPTILVTSVTSETPATSAPEAEGPQSGGLPPPPRAVPSSSSPQAQALTEDGRPCRFPFRYGGRMLHACTSEGSAHRKWCATTHNYDRDRAWGYCVEATPPPGGPAALDPCASGPCLNGGSCSNTQDPQSYHCSCPRAFTGKDCGTEKCFDETRYEYLEGGDRWARVRQGHVEQCECFGGRTWCEGTRHTACLSSPCLNGGTCHLIVATGTTVCACPPGFAGRLCNIEPDERCFLGNGTGYRGVASTSASGLSCLAWNSDLLYQELHVDSVGAAALLGLGPHAYCRNPDNDERPWCYVVKDSALSWEYCRLEACESLTRVQLSPDLLATLPEPASPGRQACGRRHKKRTFLRPRIIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIRPPRRLVAPS

SEQ ID NO: 39 - protein, 440 aa, Homo sapiens
MGPPGSPWQWVTLLLGLLLPPAAPFWLLNVLFPPHTTPKAELSNHTRPVILVPGCLGNQLEAKLDKPDVVNWMCYRKTEDFFTIWLDLNMFLPLGVDCWIDNTRVVYNRSSGLVSNAPGVQIRVPGFGKTYSVEYLDSSKLAGYLHTLVQNLVNNGYVRDETVRAAPYDWRLEPGQQEEYYRKLAGLVEEMHAAYGKPVFLIGHSLGCLHLLYFLLRQPQAWKDRFIDGFISLGAPWGGSIKPMLVLASGDNQGIPIMSSIKLKEEQRITTTSPWMFPSRMAWPEDHVFISTPSFNYTGRDFQRFFADLHFEEGWYMWLQSRDLLAGLPAPGVEVYCLYGVGLPTPRTYIYDHGFPYTDPVGVLYEDGDDTVATRSTELCGLWQGRQPQPVHLLPLHGIQHLNMVFSNLTLEHINAILLGAYRQGPPASPTASPEPPPPE

SEQ ID NO: 40 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 41 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 42 - protein, 599 aa, Homo sapiens
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 43 - protein, 396 aa, Homo sapiens [sequence omitted]

SEQ ID NO: 44 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 45 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 46 - protein, 602 aa, Homo sapiens
MHSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 47 - protein, 491 aa, Homo sapiens
MALLWGLLVLSWSCLQGPCSVFSPVSAMEPLGRQLTSGPNQEQVSPLTLLKLGNQEPGGQTALKSPPGVCSRDPTPEQTHRLARAMMAFTADLFSLVAQTSTCPNLILSPLSVALALSHLALGAQNHTLQRLQQVLHAGSGPCLPHLLSRLCQDLGPGAFRLAARMYLQKGFPIKEDFLEQSEQLFGAKPVSLTGKQEDDLANINQWVKEATEGKIQEFLSGLPEDTVLLLLNAIHFQGFWRNKFDPSLTQRDSFHLDEQFTVPVEMMQARTYPLRWFLLEQPEIQVAHFPFKNNMSFVVLVPTHFEWNVSQVLANLSWDTLHPPLVWERPTKVRLPKLYLKHQMDLVATLSQLGLQELFQAPDLRGISEQSLVVSGVQHQSTLELSEVGVEAAAATSIAMSRMSLSSFSVNRPFLFFIFEDTTGLPLFVGSVRNPNPSAPRELKEQQDSPGNKDFLQSLKGFPRGDKLFGPDLKLVPPMEEDYPQFGSPK

SEQ ID NO: 48 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 49 - protein, 202 aa, Homo sapiens
MELWGAYLLLCLFSLLTQVTTEPPTQKPKKIVNAKKDVVNTKMFEELKSRLDTLAQEVALLKEQQALQTVCLKGTKVHMKCFLAFTQTKTFHEASEDCISRGGTLSTPQTGSENDALYEYLRQSVGNEAEIWLGLNDMAAEGTWVDMTGARIAYKNWETEITAQPDGGKTENCAVLSGAANGKWFDKRCRDQLPYICQFGIV

SEQ ID NO: 50 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 51 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 52 - protein, 617 aa, Homo sapiens
MPALSRWASLPGPSMREAAFMYSTAVAIFLVILVAALQGSAPRESPLPYHIPLDPEGSLELSWNVSYTQEAIHFQLLVRRLKAGVLFGMSDRGELENADLVVLWTDGDTAYFADAWSDQKGQIHLDPQQDYQLLQVQRTPEGLTLLFKRPFGTCDPKDYLIEDGTVHLVYGILEEPFRSLEAINGSGLQMGLQRVQLLKPNIPEPELPSDACTMEVQAPNIQIPSQETTYWCYIKELPKGFSRHHIIKYEPIVTKGNEALVHHMEVFQCAPEMDSVPHFSGPCDSKMKPDRLNYCRHVLAAWALGAKAFYYPEEAGLAFGGPGSSRYLRLEVHYHNPLVIEGRNDSSGIRLYYTAKLRRFNAGIMELGLVYTPVMAIPPRETAFILTGYCTDKCTQLALPPSGIHIFASQLHTHLTGRKVVTVLVRDGREWEIVNQDNHYSPHFQEIRMLKKVVSVHPGDVLITSCTYNTEDRELATVGGFGILEEMCVNYVHYYPQTQLELCKSAVDAGFLQKYFHLINRFNNEDVCTCPQASVSQQFTSVPWNSFNRDVLKALYSFAPISMHCNKSSAVRFQGEWNLQPLPKVISTLEEPTPQCPTSQGRSPAGPTVVSIGGGKG

SEQ ID NO: 53 - protein, 591 aa, Homo sapiens
MKNSRTWAWRAPVELFLLCAALGCLSLPGSRGERPHSFGSNAVNKSFAKSRQMRSVDVTLMPIDCELSSWSSWTTCDPCQKKRYRYAYLLQPSQFHGEPCNFSDKEVEDCVTNRPCRSQVRCEGFVCAQTGRCVNRRLLCNGDNDCGDQSDEANCRRIYKKCQHEMDQYWGIGSLASGINLFTNSFEGPVLDHRYYAGGCSPHYILNTRFRKPYNVESYTPQTQGKYEFILKEYESYSDFERNVTEKMASKSGFSFGFKIPGIFELGISSQSDRGKHYIRRTKRFSHTKSVFLHARSDLEVAHYKLKPRSLMLHYEFLQRVKRLPLEYSYGEYRDLFRDFGTHYITEAVLGGIYEYTLVMNKEAMERGDYTLNNVHACAKNDFKIGGAIEEVYVSLGVSVGKCRGILNEIKDRNKRDTMVEDLVVLVRGGASEHITTLAYQELPTADLMQEWGDAVQYNPAIIKVKVEPLYELVTATDFAYSSTVRQNMKQALEEFQKEVSSCHCAPCQGNGVPVLKGSRCDCICPVGSQGLACEVSYRKNTPIDGKWNCWSNWSSCSGRRKTRQRQCNNPPPQNGGSPCSGPASETLDCS

SEQ ID NO: 54 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 55 - protein, 381 aa, Homo sapiens
MWRSLGLALALCLLPSGGTESQDQSSLCKQPPAWSIRDQDPMLNSNGSVTVVALLQASCYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSCCCHCRHLIFEKTGSAITCQCKENLPSLCSCQGLRAEENITESCQCRLPPAACQISQQLIPTEASASCRCKNQAKKCECPSN

SEQ ID NO: 56 - protein, 646 aa, Homo sapiens
MGLPRLVCAFLLAACCCCPRVAGVPGEAEQPAPELVEVEVGSTALLKCGLSQSQGNLSHVDWFSVHKEKRTLIFRVRQGQGQSEPGEYEQRLSLQDRGATLALTQVTPQDERIFLCQGKRPRSQEYRIQLRVYKAPEEPNIQVNPLGIPVNSKEPEEVATCVGRNGYPIPQVIWYKNGRPLKEEKNRVHIQSSQTVESSGLYTLQSILKAQLVKEDKDAQFYCELNYRLPSGNHMKESREVTVPVFYPTEKVWLEVEPVGMLKEGDRVEIRCLADGNPPPHFSISKQNPSTREAEEETTNDNGVLVLEPARKEHSGRYECQGLDLDTMISLLSEPQELLVNYVSDVRVSPAAPERQEGSSLTLTCEAESSQDLEFQWLREETGQVLERGPVLQLHDLKREAGGGYRCVASVPSIPGLNRTQLVNVAIFGPPWMAFKERKVWVKENMVLNLSCEASGHPRPTISWNVNGTASEQDQDPQRVLSTLNVLVTPELLETGVECTASNDLGKNTSILFLELVNLTTLTPDSNTTTGLSTSTASPHTRANSTSTERKLPEPESRGVVIVAVIVCILVLAVLGAVLYFLYKKGKLPCRRSGKQEITLPPSRKSELVVEVKSDKLPEEMGLLQGSSGDKRAPGDQGEKYIDLRH

SEQ ID NO: 57 - protein, 190 aa, Homo sapiens
MATHHTLWMGLALLGVLGDLQAAPEAQVSVQPNFQQDKFLGRWFSAGLASNSSWLREKKAALSMCKSVVAPATDGGLNLTSTFLRKNQCETRTMLLQPAGSLGSYSYRSPHWGSTYSVSVVETDYDQYALLYSQGSKGPGEDFRMATLYSRTQTPRAELKEKFTAFCKAQGFTEDTIVFLPQTDKCMTEQ

SEQ ID NO: 58 - protein, 261 aa, Homo sapiens
MASPDWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 59 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 60 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 61 - protein, 245 aa, Homo sapiens
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTQGDEAEAGEGGEN

SEQ ID NO: 62 - protein, 525 aa, Homo sapiens
MKALIAALLLITLQYSCAVSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 63 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 64 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 65 - protein, 372 aa, Homo sapiens [sequence omitted]

SEQ ID NO: 66 - protein, 313 aa, Homo sapiens
MELDRAVGVLGAATLLLSFLGMAWALQAADTCPEVKMVGLEGSDKLTILRGCPGLPGAPGPKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 67 - protein, 791 aa, Homo sapiens
MWNMLIVAMCLALLGCLQAQELQGHVSIILLGATGDLAKKYLWQGLFQLYLDEAGRGHSFSFHGAALTAPKQGQELMAKALESLSCPKDMAPSHCAEHKDQFLQLSQYRQLKTAEDYQALNKDIEAQLQHAGLREAGRIFYFSVPPFAYEDIARNINSSCRPGPGAWLRVVLEKPFGHDHFSAQQLATELGTFFQEEEMYRVDHYLGKQAVAQILPFRDQNRKALDGLWNRHHVERVEIIMKETVDAEGRTSFYEEYGVIRDVLQNHLTEVLTLVAMELPHNVSSAEAVLRHKLQVFQALRGLQRGSAVVGQYQSYSEQVRRELQKPDSFHSLTPTFAAVLVHIDNLRWEGVPFILMSGKALDERVGYARILFKNQACCVQSEKHWAAAQSQCLPRQLVFHIGHGDLGSPAVLVSRNLFRPSLPSSWKEMEGPPGLRLFGSPLSDYYAYSPVRERDAHSVLLSHIFHGRKNFFITTENLLASWNFWTPLLESLAHKAPRLYPGGAENGRLLDFEFSSGRLFFSQQQPEQLVPGPGPAPMPSDFQVLRAKYRESPLVSAWSEELISKLANDIEATAVRAVRRFGQFHLALSGGSSPVALFQQLATAHYGFPWAHTHLWLVDERCVPLSDPESNFQGLQAHLLQHVRIPYYNIHPMPVHLQQRLCAEEDQGAQIYAREISALVANSSFDLVLLGMGADGHTASLFPQSPTGLDGEQLVVLTTSPSQPHRRMSLSLPLINRAKKVAVLVMGRMKREITTLVSRVGHEPKKWPISGVLPHSGQLVWYMDYDAFLG

SEQ ID NO: 68 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 69 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA